(19) United States
(12) Patent Application Publication
(10) Pub. No.: US 2023/0390228 A1
(43) Pub. Date: Dec. 7, 2023

(54) TREATMENT METHODS USING GHB

(71) Applicant: Jazz Pharmaceuticals Ireland Limited, Dublin (IE)

(72) Inventors: Franck SKOBIERANDA, Palo Alto, CA (US); Mark Toddman KIRBY, Palo Alto, CA (US)

(73) Assignee: Jazz Pharmaceuticals Ireland Limited, Dublin (IE)

(21) Appl. No.: 18/031,994

(22) PCT Filed: Oct. 18, 2021
(86) PCT No.: PCT/US2021/055426

§ 371 Date: Apr. 14, 2023

Related U.S. Application Data
(60) Provisional application No. 63/092,833, filed on Oct. 16, 2020.

(51) Int. Cl. A61K 31/191; A61P 25/18

(52) U.S. Cl. CPC A61K 31/191; A61P 25/18

(57) ABSTRACT

Provided herein are methods of treating fibromyalgia, post-traumatic stress disorder, irritable bowel syndrome, and irritable bowel disease in a patient with a slow wave sleep deficit by administering oxybate or a pharmaceutically acceptable salt thereof.

### CROSS-REFERENCE TO RELATED APPLICATIONS

This application claims priority to U.S. Application No. 63/092,833, filed Oct. 16, 2020, which is hereby incorporated by reference in its entirety herein.

### BACKGROUND

Gamma-hydroxybutyrate (GHB), also known as “oxybate,” is an endogenous compound that is found in many human body tissues. GHB is present, for example, in the mammalian brain and other tissues. In the brain, the highest GHB concentration is found in the hypothalamus and basal ganglia and GHB is postulated to function as an inhibitory neurotransmitter (Snead and Morley, 1981, Brain Res. 227(4): 579-89). The neuropharmacologic effects of GHB include increases in brain acetylcholine, increases in brain dopamine, inhibition of GABA-ketoglutarate transaminase and depression of glucose utilization, but not oxygen consumption in the brain. GHB treatment substantially reduces the signs and symptoms of narcolepsy, i.e., daytime sleepiness, cataplexy, sleep paralysis, and hypnagogic hallucinations. In addition, GHB increases total sleep time and REM sleep, and it decreases REM latency, reduces sleep apnea, and improves general anesthesia (see U.S. Pat. Nos. 6,472,431; 6,780,889; 7,262,219; 7,851,506; 8,263,650; and 8,324,275, the disclosure of each of which is incorporated by reference in its entirety for all purposes).

Oxybate has been reported to be effective for relieving pain and improving function in a number of conditions. Because of its therapeutic potential, there is need in the art for methods of identifying and targeting specific patient subpopulations who may benefit from oxybate therapy.

### SUMMARY

The present disclosure provides methods of identifying patients who would benefit from oxybate treatment using a patient's slow wave sleep as an indicator for oxybate response enrichment. The present disclosure further provides methods of treating fibromyalgia, post-traumatic stress disorder, irritable bowel syndrome, and irritable bowel disease in a patient with a slow wave sleep deficit.

In one aspect, the present disclosure provides a method of treating the symptoms associated with fibromyalgia in a patient in need thereof, the method comprising:
(a) identifying a patient with fibromyalgia with a slow wave sleep (SWS) deficit as determined by polysomnography; and
(b) administering a therapeutically effective amount of oxybate or a pharmaceutically acceptable salt thereof to the patient.

In one aspect, the present disclosure provides a method of treating the symptoms associated with fibromyalgia in a patient in need thereof, the method comprising:
(a) identifying a patient with fibromyalgia with a slow wave sleep (SWS) deficit as determined by polysomnography (e.g. sleep electroencephalogram (EEG)); and
(b) administering a therapeutically effective amount of oxybate or a pharmaceutically acceptable salt thereof to the patient.

In some embodiments of the methods of the present disclosure, the administration provides an improvement in the patient's Fibromyalgia Impact Questionnaire (FIQ) score compared to prior to the treatment, Tender Points Index (TPI) score compared to prior to the treatment, Pain Visual Analogue Scale (P-VAS) score compared to prior to the treatment, Fatigue Visual Analogue Scale (F-VAS) score compared to prior to the treatment, or Tender Points Count (TPC) score compared to prior to the treatment.

In some embodiments, the present disclosure provides a method of treating the symptoms associated with post-traumatic stress disorder (PTSD) in a patient in need thereof, the method comprising:
(a) identifying a patient with PTSD with a SWS deficit as determined by polysomnography (e.g., sleep EEG); and
(b) administering a therapeutically effective amount of oxybate or a pharmaceutically acceptable salt thereof.

In some embodiments of the methods of the present disclosure, the identified patient exhibits a SWS deficit. In some embodiments the identified patient exhibits SWS of less than about 15%, less than about 10%, less than about 5% or less than about 1%.

In some embodiments of the methods of the present disclosure, methods of the present disclosure provide an improvement in the patient's sleep-related PTSD symptoms (e.g., insomnia, nightmares and somniphobia) compared to prior to the treatment.

In some embodiments of the methods of the present disclosure, the oxybate or a pharmaceutically acceptable salt thereof is a mixed salt oxybate. In some embodiments, the oxybate or a pharmaceutically acceptable salt comprises sodium oxybate.

In some embodiments, the method comprises:
(a) administering an initial daily dose of the oxybate or a pharmaceutically acceptable salt thereof to the patient and
(b) titrating the dose to provide a therapeutically effective amount of the oxybate or a pharmaceutically acceptable salt thereof.

In some embodiments of the methods of the present disclosure, the oxybate or a pharmaceutically acceptable salt thereof composition is a liquid.

In some embodiments of the methods of the present disclosure, the oxybate composition is an oral formulation which provides sustained release of the oxybate or a pharmaceutically acceptable salt thereof.

In some embodiments of the methods of the present disclosure, the oxybate composition is an immediate-release/modified release (IR/MR) formulation of the oxybate or a pharmaceutically acceptable salt thereof.

### Definitions

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range. Equally

The terms “administer,” “administering” or “administration” as used herein refer to administering a compound or pharmaceutically acceptable salt of the compound or a composition or formulation comprising the compound or pharmaceutically acceptable salt of the compound to a patient.

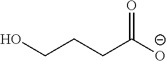

hydroxybutyrate. GBA has the following structural formula:

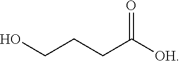

Salt forms of GHB are disclosed in U.S. Pat. Nos. 8,591,922; 8,901,173; 9,132,107; 9,555,017; and 10,195,168, which are hereby incorporated by reference in their entireties for all purposes.

The terms “effective amount” and “therapeutically effective amount” are used interchangeably in this disclosure and refer to an amount of a compound, or a salt thereof, that, when administered to a patient, is capable of performing the intended result. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The term “equivalent” when comparing Na·GHB and mixed salts forms contains the same amount of GHB within about 5% (by weight %). In preferred embodiments, a liquid formulation of a mixed salt is equivalent to the Na·GHB-containing liquid formulation Xyrem (which contains 0.409 g/mL of GHB).

In preferred embodiments, a liquid formulation of a mixed salt contains 0.234 g/mL of calcium oxybate, 0.130 g/mL of potassium oxybate, 0.096 g/mL of magnesium oxybate, and 0.040 g/mL of sodium oxybate.

As used herein, the term “patient” refers to a mammal, particularly a human.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “carrier” encompasses solvents, dispersion media, coatings, antibacterial and antifungal agents, isotonic and absorption delaying agents and the like. The use of carriers for active pharmaceutical ingredients is well known in the art. Insofar as any conventional media or agent is incompatible with the active ingredient, its use in the therapeutic compositions is not appropriate.

The term “therapeutic effect” as used herein refers to a desired or beneficial effect provided by the method and/or the composition. In some embodiments, the methods of the present disclosure provide a therapeutic effect when the method improves at least one symptom of the patient's disease or disorder (such as fibromyalgia, PTSD, IBD or IBS) as measured by a validated clinical test (such as those known to those skilled in the art or as described herein). For example, the method for treating fibromyalgia in a patient with a slow wave sleep (SWS) deficit provides a therapeutic effect when the method reduces at least one symptom of fibromyalgia, such as widespread pain in soft tissue regions like muscles, ligaments, and tendons. Likewise, the method for treating PTSD in a patient with a slow wave sleep (SWS) deficit provides a therapeutic effect when the method reduces at least one symptom of PTSD.

The term “treating” as used herein with regard to a patient, refers to improving at least one symptom of the patient's disorder. Treating can be improving, or at least partially ameliorating a disorder. The terms “substitute”, “switch”, “change”, “transition” and “exchange” are used interchangeably in the context of the present disclosure.

The term “salt” or “salts,” as used herein, refers to a compound formed by the interaction of an acid and a base, the hydrogen atoms of the acid being replaced by the positive ion or cation of the base. Pharmaceutically acceptable salts include inorganic acids such as, for example, hydrochloric or phosphoric acids, or such organic acids as malic, acetic, oxalic, tartaric, mandelic, and the like. Salts formed can also be derived from inorganic bases such as, for example, sodium, potassium, silicates, ammonium, calcium, or ferric hydroxides, and such organic bases as isopropylamine, trimethylamine, histidine, procaine and the like. In certain preferred embodiments, the salt is formed from an inorganic base that is a metal, for example, an alkali metal, such as lithium, potassium, sodium, or the like, an alkaline earth metal, such as magnesium, calcium, barium, or the like, or aluminum or zinc. Other salts may comprise ammonium. Alkali metals, such as lithium, potassium, sodium, and the like, may be used, preferably with an acid to form a pH adjusting agent. Examples of pharmaceutically acceptable base addition salts include those derived from inorganic bases like sodium hydroxide, potassium hydroxide, magnesium hydroxide, calcium hydroxide, or ammonium hydroxide, and the like (See, e.g., Berge et al., 1977, J. Pharm. Sci. 66: 1, U.S. Pat. Nos. 6,472,431 and 8,591,922).

As used herein, the terms “salt of GHB” or “salts of GHB,” as used herein, refer to a compound formed by the interaction of gamma-hydroxybutyric acid (the conjugate acid of GHB) with a base, for example, NaOH, KOH, Mg(OH)₂, and Ca(OH)₂, and the like, the hydrogen atoms of the acid being replaced by the positive ion or cation of the base. Such salts may include, for example, sodium oxybate (“Na·GHB”), potassium oxybate (“K·GHB”), magnesium oxybate (“Mg·(GHB)₂”), and calcium oxybate (“Ca·(GHB)₂”), and the like. It will be understood by those skilled in the art that such salts may be in solid form, or such salts may be in partially or fully solvated form, for example, as when dissolved in an aqueous medium. It will be further understood by those skilled in the art, that, depending on the solubility of the salt in the aqueous medium, that the salt may be present in the aqueous medium as solvated cation(s) and anion(s), or as a precipitated solid.

The term “oxybate dosing strength” refers to the amount of GHB in a particular dose (e.g., each mL of Xyrem contains 0.5 g of sodium oxybate, which is equivalent to a 0.409 g/mL oxybate dosing strength). Although throughout the present disclosure, the oxybate dosing strength in a composition is generally expressed in terms of the amount of oxybate present in a composition, the present disclosure contemplates embodiments where the oxybate dosing strength is expressed in the Equivalent Concentration of GBA that is contained in the dose.

The Equivalent Concentration of GBA in a compositions may be calculated by the following formula:

$$Equivalent Concentration of GBA = \frac{Concentration of GHB in ( g / mL ) \times 104.1 ( Formula Weight of GBA , \frac{g}{mol} )}{103.1 ( Formula Weight of GHB ( \frac{g}{mol} )}$$

Thus, each mL of Xyrem contains 0.5 g of sodium oxybate, which is equivalent to an Equivalent Concentration of GBA of 0.413 g/mL.

The term “JZP-258” as used herein refers to a solution containing the mixed salt oxybate comprising about 8% sodium oxybate, about 23% potassium oxybate, about 21% magnesium oxybate and about 48% calcium oxybate (% mol. equiv. of GHB) and having a GHB concentration of 0.409 g/mL (or, expressed another way, an Equivalent Concentration of GBA of 0.413 g/mL). The following table describes the % mol. equiv., wt/vol %, and absolute amount of sodium oxybate, potassium oxybate, magnesium oxybate and calcium oxybate in representative doses of JZP-258.

| | % mol | | Amount in 1 g | | Amount in 9 g | |
|---|---|---|---|---|---|---|
| | equivalent | wt/wt % | JZP-258 | | JZP-258 | |
| Na. GHB | 8 | 8 | 80 | mg | 720 | mg |
| K. GHB | 23 | 25.5 | 255 | mg | 2,295 | mg |
| Mg. (GHB)₂ | 21 | 19.5 | 195 | mg | 1,755 | mg |
| Ca. (GHB)₂ | 48 | 47 | 470 | mg | 4,230 | mg |

The term “mixed salts” or “mixed salt oxybate,” as used herein, refers to salts of GHB where two, three, four or more different cations are present in combination with each other in a composition. Such mixtures of salts may include, for example, salts selected from the group consisting of Na·GHB, K·GHB, Mg·(GHB)₂, and Ca. (GHB)₂. Mixed salt oxybates are described in U.S. Pat. Nos. 8,591,922; 8,901,173; 9,132,107; 9,555,017; and 10,195,168, the contents of which is hereby incorporated by reference it entirety for all purposes.

The term “wt/wt %,” as used herein, refers to the normalized weight percent of a particular salt in a salt mixture.

The term “wt/wt % ratio,” as used herein, refers to the ratio of wt/wt % values in a mixture of salt. For example, where the salts Na·GHB, K·GHB, Mg·(GHB)₂, and Ca. (GHB)₂ are present in a wt/wt %'s of 8%, 25.5%, 19.5% and 47%, respectively, the wt/wt % ratio of Na·GHB, K·GHB, Mg·(GHB)₂, and Ca·(GHB)₂ in the mixture is 8%:25.5%:19.5%:47%.

The term, “formulation,” as used herein, refers to a stable and pharmaceutically acceptable preparation of a pharmaceutical composition disclosed herein.

The term, “liquid formulation,” as used herein, refers to a water-based formulation, in particular, a formulation that is an aqueous solution.

The term “% Slow Wave Sleep” (or “% SWS”) as used herein, refers to the percentage of the total sleep time (TST) spent in S3/S4 sleep (N3), as determined by polysomnography, or other methods known in the art or described herein that can detect slow wave sleep, such as actigraphy, and peripheral arterial tone. The terms Slow Wave Sleep (SWS), S3/S4 sleep, and N3 sleep are used interchangeably herein. In some embodiments, a patient's % Slow Wave Sleep is determined using the methods described in Iber, et al., “The AASM Manual for the Scoring of Sleep and Associated Events, American Academy of Sleep Medicine”.

The term “Minimal Clinically Important Difference” (“MCID”) as used herein refers to the smallest difference that patients and/or physicians perceive as beneficial and that would mandate, in the absence of excessive side effects, a change in the patient's management.

### BRIEF DESCRIPTION OF THE DRAWINGS

FIG. 1 shows the mean change from baseline in pain visual analogue scale and Fibromyalgia Impact Questionnaire (FIQ) in fibromyalgia patients with less than approximately 1% slow wave sleep (left) and ≥20% slow wave sleep (right) treated with placebo, 4.5 g of Xyrem and 6.0 grams of Xyrem. The data show that patients with low slow wave sleep exhibit a dose-dependent response to Xyrem in pain visual analogue scale and FIQ whereas patients with ≥20% slow wave sleep did not have a dose-dependent response.

FIG. 2A shows baseline Fibromyalgia Impact Questionnaire (FIQ) scores in fibromyalgia patients from the lowest quartile (Q1) and highest quartile (Q4) of slow wave sleep. FIG. 2B shows baseline Pain Visual Analog Scale (Pain VAS) scores in fibromyalgia patients from Q1 and Q4 of % slow wave sleep. FIG. 2C shows baseline Fatigue Visual Analog Scale (Fatigue VAS) scores in fibromyalgia patients from Q1 and Q4 of % slow wave sleep. FIG. 2D shows baseline Tender Points Count (TPC) scores in fibromyalgia patients from Q1 and Q4 of % slow wave sleep. The data show that, at baseline, patients from Q1 and Q4 of % slow wave sleep exhibit similar Fatigue VAS, TPC and FIQ scores.

FIG. 3A shows the change in FIQ from baseline in fibromyalgia patients treated with placebo, 4.5 g of Xyrem and 6.0 grams of Xyrem from slow wave sleep Q1 (left) and Q4 (right). FIG. 3B shows the change in Pain Visual Analog Scale (Pain VAS) from baseline in fibromyalgia patients treated with placebo, 4.5 g of Xyrem and 6.0 grams of Xyrem from slow wave sleep Q1 (left) and Q4 (right). FIG. 3C shows the change in Fatigue VAS from baseline in fibromyalgia patients treated with placebo, 4.5 g of Xyrem and 6.0 grams of Xyrem from slow wave sleep Q1 (left) and Q4 (right). FIG. 3D shows the change in Tender Points Count (TPC) from baseline in fibromyalgia patients treated with placebo, 4.5 g of Xyrem and 6.0 grams of Xyrem from slow wave sleep Q1 (left) and Q4 (right). The data show that oxybate-treated patients from Q1 of % slow wave sleep exhibit significant improvements in Fatigue VAS, TPC and FIQ scores whereas oxybate-treated patients from Q4 of % slow wave sleep exhibit Fatigue VAS, TPC and FIQ scores that are similar to placebo.

FIG. 4 shows representative % slow wave sleep by age (left) and by age and sex (right).

FIG. 5 shows a cluster assignment scatterplot of slow wave sleep (N3) relative to age in the “Fibromyalgia” population of Example 3.

FIG. 6 shows a scatterplot of percent slow wave sleep (N3) relative to age and biological sex in the “PTSD” population of Example 3.

FIGS. 7A-C shows the change in Pain VAS from baseline (Y-axis) versus baseline percentage N3 sleep (X-axis), for patients treated with placebo (FIG. 7A), 4.5 g Xyrem (FIG. 7B), and 6 g Xyrem (FIG. 7C).

### DETAILED DESCRIPTION

Sleep includes rapid eye movement (REM) sleep and non-rapid eye movement (Non REM or NREM) sleep, where non REM sleep can be further broken down into shallow Non REM sleep and deep Non REM sleep. The sleep cycle includes stages W (wakefulness), N1 (NREM 1), N2 (NREM 2), N3 (NREM3), and R (REM), which can be identified by polysomnography. The signals recorded in a polysomnography study include, but are not limited to brain electrical activity (electroencephalogram, or EEG); ocular muscle movements (electrooculogram, or EOG); or somatic muscle movement (electromyogram, or EMG), and combinations thereof. Six wave patterns in EEG are typically used to differentiate wake and sleep states and classify sleep stages: (1) alpha activity, (2) theta activity, (3) vertex sharp waves, (4) sleep spindles, (5) K complexes, and (6) slow wave activity. The criteria for each stage, and methods for determining the stage of a sleeping subject, and profiling a subject's sleep architecture are described in Iber, et al., “The AASM Manual for the Scoring of Sleep and Associated Events, American Academy of Sleep Medicine” and in the Rechtschaffen and Kales (R and K) sleep scoring manual of 1968, which are hereby incorporated by reference herein in their entirety for all purposes.

Stage N3 NREM sleep may also be termed deep sleep, slow wave sleep (SWS), or delta sleep. The new AASM stage N3 includes R and K stages 3 and 4 together. SWS is marked by high-amplitude slow waves. Slow waves are high-amplitude (≥75 μV) and low-frequency (≤2 Hz) variants of delta (1-4 Hz) activity, as observed by EEG. No specific criteria for EOG and EMG exist for SWS, but in general, muscle tone is further decreased. SWS constitutes the deepest, most refreshing and restorative sleep type, which tends to diminish with age.

In narcolepsy patients with cataplexy, treatment with sodium oxybate decreased the number of sleep arousals, increased consolidation of REM sleep and increased the duration of slow wave sleep.

### Fibromyalgia

Fibromyalgia, also referred to as fibromyalgia syndrome (FMS) or fibrositis syndrome, is a common chronic disease characterized by widespread pain and allodynia at 11 or more of 18 anatomically defined tender points. The widespread pain is present in soft tissue regions like muscles, ligaments, and tendons but does not involve joints. While FMS is classified by pain and tenderness, many other clinical features are present in patient subgroups. Other common clinical symptoms of FMS include chronic insomnia, prolonged morning stiffness, chronic fatigue, non-restorative sleep, recurrent headaches, anxiety, depression, cognitive impairment, and gastrointestinal (GI) symptoms that are associated with irritable bowel syndrome (IBS).

Patients with fibromyalgia report widespread musculoskeletal pain, chronic fatigue, and non-restorative sleep. These patients show specific regions of localized tenderness in the absence of demonstrable anatomic or biochemical pathology, and patients suffering from fibromyalgia typically describe light and/or restless sleep, often reporting that they awaken feeling unrefreshed with pain, stiffness, physical exhaustion, and lethargy. See, H. D. Moldofsky et al., J. Muscoloskel. Pain, 1, 49 (1993). Aspects of the patients' sleep pathology are related to their pain and mood symptoms (Moldofsky 1993). That is, patients with FMS show an alpha (7.5 to 11 Hz) electroencephalographic (EEG), non-rapid-eye-movement (NREM) sleep anomaly correlated with musculoskeletal pain and altered mood. Alpha EEG NREM sleep anomaly is an indicator of an arousal disorder within sleep associated with the subjective experience of non-restorative sleep. See H. D. Moldofsky et al., Psychosom. Tailed., 37, 341 (1975).

### Post-Traumatic Stress Disorder (PTSD)

Posttraumatic stress disorder (PTSD) involves responses to a traumatic event that persist maladaptively. PTSD is characterized by intrusive thoughts related to the event, avoidance of reminders of the event, negative mood and cognitions, and heightened arousal and reactivity. In the United States, about 3.5% of adults have PTSD in a given year, and 9% of people develop it at some point in their life. In much of the rest of the world, rates during a given year are between 0.5% and 1%.

The majority of PTSD patients have significant sleep disturbances, which independently contribute to poor daytime function, are often resistant to first-line treatment, and often require sleep-focused treatments. Sleep disturbances are also predictive of the development of PTSD. Assessments of sleep disturbances include sleep diary, self-report questionnaire, face-to-face interview, peripheral arterial tone, actigraphy, and polysomnography (PSG). Among these methods, PSG is required to distinguish rapid eye movement (REM) sleep, non-REM sleep, and stages 1-3 (N1-3) of non-REM sleep. PSG determined alterations in sleep are highly important for understanding the etiology and neurobiology of PTSD. For instance, Ross et al. (Am J Psychiatry 1989; 146(6):697e707) emphasized REM sleep disturbances and nightmares as hallmarks of PTSD, Germain et al. (Sleep Med Rev 2008; 12(3):185e95) also emphasized a role for REM sleep, and proposed that it amplifies altered function of the amygdala and medial frontal cortex in PTSD patients whereby amplification of abnormal amygdala activation in combination with reduced activation of the medial prefrontal cortex could subserve nightmares. Zhang et al. (Sleep Medicine Reviews 48 (2019) 101210) found, that patients with posttraumatic stress disorder exhibit disturbances in sleep architecture and sleep continuity compared to healthy controls. The study suggested that total sleep time, slow wave sleep (SWS), and sleep efficiency (SE) were decreased and WASO increased in patients with PTSD compared with healthy controls. The study also found that PTSD severity as evaluated by CAPS was associated with decreased SE and SWS percentage.

### Irritable Bowel Disease (IBD) and Irritable Bowel Syndrome (IBS)

Irritable bowel syndrome (IBS) is characterized by chronic, recurring abdominal discomfort or pain with impaired bowel habits that cannot be explained by structural or biochemical abnormalities. IBS is the most common diagnosis made by gastroenterologists in the United States with a lifetime prevalence ranging from 8% to 20% among adults. Irritable bowel disease (IBD) is a term for two conditions (Crohn's disease and ulcerative colitis) that are characterized by chronic inflammation of the gastrointestinal tract. In 2015, an estimated 1.3% of US adults were diagnosed with IBD.

Sleep disturbances are commonly reported in IBS and IBD patients. Rotem et al. (Rotem et al. Sleep, Vol. 26, No. 6, 2003) compared the sleep of IBS patients to a matched comparison group. The study showed that IBS patients suffer from a considerable degree of sleep fragmentation, including an elevated arousal index, more shallow sleep, and prolonged awakening during sleep. Symptoms indicative of IBS severity, such as pain and Functional Bowel Disorder Severity Index (FBDSI) correlated significantly with sleep fragmentation. Compared to normal subject, the sleep of IBS patients was lighter and IBS patients had a significant increase in cumulative as well as continuous stage 2 (NREM 2) sleep. Patients with IBS also exhibited a reduction in SWS stage, which was more than 50% lower than the matched comparison group. Patients with IBS also had more frequent arousals and awakenings, frequent sleep-stage shifts, and waking periods during sleeps than the comparison group. Patients with IBS also exhibited symptoms of daytime sleepiness as reflected in the higher scores on the Epworth Sleepiness Scale in the IBS group.

Taft evaluated the frequency of PTSD in IBD patients (Taft et al. Inflamm. Bowel Dis, Vol. 25, No. 9, 2019) and found that approximately one-third of IBD patients reported significant PTSD symptoms and one-quarter reported a clinical PTSD diagnosis since disease onset.

Thus, there is considerable sleep symptom overlap between fibromyalgia, PTSD, IBS and IBD.

In one aspect, the present disclosure provides methods of treating fibromyalgia in a patient in need thereof by administering an effective amount of oxybate and, in particular, to a fibromyalgia patient who exhibits a slow wave sleep deficit.

In one aspect, the present disclosure provides methods of treating PTSD in a patient in need thereof by administering an effective amount of oxybate and, in particular, to a PTSD patient who exhibits a slow wave sleep deficit.

In one aspect, the present disclosure provides methods of treating IBS or IBD in a patient in need thereof by administering an effective amount of oxybate and, in particular, to an IBS or MD patient who exhibits a slow wave sleep deficit.

In one aspect, the present disclosure provides methods of treating IBS in a patient in need thereof by administering an effective amount of oxybate and, in particular, to an IBS patient who exhibits a slow wave sleep deficit.

In one aspect, the present disclosure provides methods of treating TBD in a patient in need thereof by administering an effective amount of oxybate and, in particular, to an MD patient who exhibits a slow wave sleep deficit.

### Sodium Oxybate

Sodium oxybate (Na·GHB), commercially sold as Xyrem®, is approved for the treatment of cataplexy or excessive daytime sleepiness in patients 7 years of age or older with narcolepsy.

Administration of the approved daily dose of Xyrem® (6-9 grams per night administered orally) results in the patient ingesting from 1100-1638 mg of sodium daily. The American Heart Association has recommended a daily sodium intake of less than 2300 mg and an “ideal” daily intake of <1500 mg (AHA 2017 (https://www.heart.org/-/media/data-import/downloadables/8/2/0/pe-abh-why-should-i-limit-sodium-ucm_300625.pdf); Whelton et al. (2012).

### Mixed Salt Oxybate

JZP-258 was developed to provide the same treatment benefits as Xyrem with substantially less sodium.

JZP-258 is a mixed salt oxybate that contains calcium oxybate, magnesium oxybate, potassium oxybate, and sodium oxybate, and it provides 87-131 mg of sodium when administered in the dose range of 6-9 grams nightly. This amount is 92% less sodium than that provided by Xyrem® administration at an equivalent dose.

The following patents, publications and application are related to the present disclosure and are hereby incorporated by reference in their entireties for all purposes: U.S. Pat. Nos. 6,472,431; 6,780,889; 7,262,219; 8,263,650; 8,461,203; 8,859,619; 9,539,330; 7,851,506; 8,324,275; 8,952,062; 8,731,963; 8,772,306; 8,952,029; 9,050,302; 9,486,426; 10,213,400; 8,591,922; 8,901,173; 9,132,107; 9,555,017; 10,195,168; 8,778,301; 9,801,852; 8,771,735; 8,778,398; 9,795,567; U.S. Patent Publication Nos. US 2020/0330393, 2018/0042855 and 2012/0076865.

In some embodiments, the methods of the present disclosure comprise administering sodium oxybate or a mixed salt oxybate to a patient in need thereof (e.g., a fibromyalgia or PTSD patient with a slow wave sleep deficit). In some embodiments, the mixed salt oxybate comprises gamma-hydroxybutyrate (GHB) and three or four or more pharmaceutically acceptable cations of an alkali metal or an alkaline earth metal. In some embodiments, the mixed salt oxybate comprises GHB and more than one pharmaceutically acceptable cations of an alkali metal or an alkaline earth metal.

In some embodiments, the mixed salt oxybate comprises GHB and two, three, or four cations selected from the group consisting of Na⁺, K⁺, Mg²⁺, and Ca²⁺. In some embodiments, mixed salt oxybate comprises GHB and all three cations selected from the group consisting of K⁺, Mg²⁺, and Ca²⁺. In some embodiments, the mixed salt oxybate does not contain Na⁺, or comprises less than 100% of Na t.

In some embodiments, the mixed salt oxybate comprises two, three, or four salts selected from the group consisting of a sodium salt of hydroxybutyrate (Na·GHB), a potassium salt of gamma-hydroxybutyrate (K·GHB), a magnesium salt of gamma-hydroxybutyrate (Mg·(GHB)₂), and a calcium salt of gamma-hydroxybutyrate (Ca·(GHB)₂). In some embodiments, the mixed salt oxybate comprises varying weight/weight percentages (wt/wt %) of Na·GHB, K·GHB, Mg·(GHB)₂, and Ca·(GHB)₂.

In some embodiments, any of the salts, such as the Na·GHB salt, the K·GHB salt, the Mg·(GHB)₂ salt or the Ca·(GHB)₂, is present in about 1% to about 100% (wt/wt %), including about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, to about 100%, including any subrange or value therebetween.. In some embodiments, the Na·GHB salt is present in a wt/wt % of about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% (wt/wt %). In some embodiments, the Na·GHB salt is absent.

In some embodiments, where the mixed salt oxybate comprises a mixture of Na·GHB, K·GHB, Mg·(GHB)₂, and Ca·(GHB)₂, the Na·GHB salt is present in a wt/wt % of about 1%-15%, 5%-10%, or about 8%; the K·GHB salt is present in a wt/wt % of about 10%-30%, 15%-25%, or about 25.5%; the Mg·(GHB)₂ salt is present in a wt/wt % of about 10%-30%, 15%-25%, or about 19.5%; and the Ca·(GHB)₂ salt is present in a wt/wt % of about 30%-60%, 40%-50, or about 47% (wt/wt %).

In some embodiments, the mixed salt oxybate comprises about 8% of sodium oxybate (wt/wt %), about 25.5% of potassium oxybate (wt/wt %), about 19.5% of magnesium oxybate (wt/wt %) and about 47% of calcium oxybate (wt/wt %). In some embodiments, where the mixed salt oxybate comprises a mixture of Na·GHB, K·GHB, Mg·(GHB)₂, and Ca·(GHB)₂, the Na·GHB, K·GHB, Mg·(GHB)₂, and Ca·(GHB)₂ salts are present in a wt/wt % ratio of about 8:25.5:19.5:47, respectively.

In some embodiments, a mixed salt oxybate of the present disclosure is dissolved in a liquid (such as water) to provide a pharmaceutical composition and the concentration of the mixed salt oxybate is expressed in terms of the wt/vol %. In some embodiments, where the mixed salt oxybate comprises a mixture of Na·GHB, K·GHB, Mg·(GHB)₂, and Ca·(GHB)₂, the Na·GHB salt is present in a wt/vol % of about 1% to about 15%, including about 1%, about 2%, about 3%, about 4%, about 5%, about 6%, about 7%, about 8%, about 9%, about 10%, about 11%, about 12%, about 13%, about 14%, to about 15% (wt/vol %), including any subrange or value therebetween; the K·GHB salt is present in a wt/vol % of about 10%-30%, including about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, to about 30% (wt/vol %) including any subrange or value therebetween; the Mg·(GHB)₂ salt is present in a wt/vol % of about 10%-30%,including about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, to about 30% (wt/vol %), including any subrange or value therebetween; and the Ca·(GHB)₂ salt is present in a wt/vol % of about 30%-60%, including about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, to about 60% (wt/vol %) including any subrange or value therebetween.

In some embodiments, the liquid pharmaceutical composition containing the mixed salt oxybate comprises about 8% of sodium oxybate (wt/vol %), about 26.0% of potassium oxybate (wt/vol %), about 19.2% of magnesium oxybate (wt/vol %) and about 46.8% of calcium oxybate (wt/vol %).

In some embodiments, the mixed salt oxybate comprises varying percentages of oxybate, expressed as % molar equivalents (% mol. equiv.) of Na·GHB, K·GHB, Mg·(GHB)₂, and Ca·(GHB)₂. The terms “% molar equivalents” and “% mol. equiv.,” as used herein, refer to molar composition of salts expressed as a percent of GHB equivalents. Those skilled in the art will understand that as each GHB unit is considered to be one molar equivalent, the monovalent cations, Na⁺ and K⁺, have one molar equivalent per salt, and the divalent cations, Mg²⁺ and Ca²⁺, have two molar equivalents per salt. See U.S. Pat. Nos. 8,591,922; 8,901,173; 9,132,107; 9,555,017; for amounts of % mol. equiv. useful in the present disclosure.

In some embodiments, any of the salts, such as the Na·GHB salt, the K·GHB salt, the Mg·(GHB)₂ salt or the Ca·(GHB)₂, is present in about 1% to about 100% (% mol. equiv), including about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, to about 100%, including any subrange or value therebetween. In some embodiments, the Na·GHB salt is present in a % mol. equiv. of about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% (% mol. equiv.). In some embodiments, the Na·GHB salt is absent.

In some embodiments, where the mixed salt oxybate comprises a mixture of Na. K·GHB, Mg·(GHB)₂, and Ca·(GHB)₂, the Na·GHB salt is present in a % mol. equiv. of about 1%-15%, 5%-10%, or about 8%; the K·GHB salt is present in a % mol. equiv. of about 10%-30%, 15%-25%, or about 23%; the Mg·(GHB)₂ salt is present in a % mol. equiv. of about 10%-30%, 15%-25%, or about 21%; and the Ca·(GHB)₂ salt is present in a % mol. equiv. of about 30%-60%, 40%-50, or about 48% (% mol. equiv.).

In some embodiments, the mixed salt oxybate comprises about 8% mol. equiv. of sodium oxybate, about 23% mol. equiv. of potassium oxybate, about 21% mol. equiv. of magnesium oxybate and about 48% mol. equiv. of calcium oxybate. In some embodiments, where the mixed salt oxybate comprises a mixture of Na·GHB, K·GHB, Mg·(GHB)₂, and Ca·(GHB)₂, wherein the mixture comprises Na·GHB, K·GHB, Mg·(GHB)₂, and Ca·(GHB)₂ salts are present in a % mol. equiv. ratio of about 8:23:21:48, respectively.

In some embodiments, where the pharmaceutical composition comprises a mixture of Na·GHB, K·GHB, and Ca·(GHB)₂, the Na·GHB salt is present in a % mol. equiv. of about 5%-40%, the K·GHB salt is present in a % mol. equiv. of about 10%-40%, and the Ca·(GHB)₂ salt is present in a % mol. equiv. of about 20%-80%.

### Pharmaceutical Compositions:

In some embodiments, the oxybate or a pharmaceutically acceptable salt thereof is in the form of a pharmaceutical composition that is suitable for administration in the methods of the present disclosure. In some embodiments, the pharmaceutical composition comprises an aqueous solution. Other formulations can be solid formulations.

In some embodiments, the concentration of a salt or mixture of salts of GHB in the liquid solution is about 50 mg/mL-950 mg/mL, about 250 mg/mL-750 mg/mL, about 350 mg/mL-650 mg/mL, or about 450 mg/mL-550 mg/mL. In some embodiments, the concentration of the salt or mixture of salts of GHB in the solution is about 500 mg/mL. In some embodiments, the pH of the pharmaceutical composition is about 7.0-9.0, about 7.0-8.5, or about 7.3-8.5.

In some embodiments, the pharmaceutical composition is chemically stable and resistant to microbial growth. In some embodiments, the pharmaceutical composition does not need, and is free of preservatives. See U.S. Pat. Nos. 6,472,431; 6,780,889; 7,262,219; 8,263,650; 8,461,203 and others for a relationship between pH and GHB concentration and their effect on microbial growth.

In some embodiments, a pH adjusting or buffering agent may be added to the pharmaceutical composition. The choice of a pH adjusting or buffering agent may affect the resistance to microbial challenge and/or the stability of GHB, as measured by the reduction in assayable GHB. Pharmaceutical compositions of GHB, pH adjusted or buffered with malic acid are resistant to both microbial growth and chemical degradation of GHB, and are preferred. Other pH adjusting or buffering agents may be selected. Agents that adjust pH that are selected on this basis will undergo a taste testing study. However, any pH adjusting or buffering agent disclosed herein or as would be known to those skilled in the art is contemplated as being useful from the compositions or formulations disclosed herein. Of course, any salt, flavoring agent, excipient, or other pharmaceutically acceptable addition described herein or as would be known to those skilled in the art is contemplated as being useful for the compositions or formulations disclosed herein.

In some embodiments, the pH adjusting or buffering agent is an acid. In some embodiments, the pH adjusting or buffering agent is an inorganic acid or an organic acid. In some embodiments, the pH adjusting or buffering agent is selected from the group consisting of malic acid, citric acid, acetic acid, boric acid, lactic acid, hydrochloric acid, phosphoric acid, sulfuric acid, sulfonic acid, and nitric acid. In some embodiments, the pH adjusting or buffering agent is malic acid. See U.S. Pat. No. 6,472,431.

The aqueous solutions disclosed herein typically comprise an effective amount of GHB, which may be dissolved or dispersed in a pharmaceutically acceptable carrier and/or an aqueous medium.

In some embodiments, the oxybate or a pharmaceutically acceptable salt thereof is in a liquid. In some embodiments, the concentration of the oxybate or a pharmaceutically acceptable salt thereof in the liquid is about 0.5 g/mL. In other embodiments, oxybate or a pharmaceutically acceptable salt thereof is a solid.

### Formulations

In some embodiments, the pharmaceutical compositions disclosed herein are provided in a formulation that is suitable for administration in the methods of the present disclosure.

In some embodiments, the formulation is a liquid formulation. In some embodiments, the formulation is a solid formulation. In some embodiments, the formulation is suitable for oral administration. See incorporated by reference U.S. Pat. Nos. 6,472,431; 6,780,889; 7,262,219; 8,263,650; 8,461,203, 8,591,922, 8,901,173, 9,132,107, 9,555,017, 9,795,567, 10,195,168, U.S. Ser. Nos. 16/688,797, 62/769,380 and 62/769,382, U.S. Patent Publication Nos. 2020/0330393, 2018/0263936 and 2012/0076865 for example. These patents and applications present examples of flavoring agents, sweeteners, coloring agents, surfactants, carriers, excipients, binders, buffering compounds or agents and other formulation ingredients.

In some embodiments, the formulation is chemically stable and resistant to microbial growth. In some embodiments, the formulation is free of preservatives. In some embodiments, the level of gamma-butyrolactone (GBL) is 0.1% or less of the formulation.

In some embodiments, the formulation is resistant to alcohol-induced dose dumping.

In preferred embodiments, the formulation is a liquid formulation, wherein the formulation comprises 0.234 g/mL of calcium oxybate, 0.130 g/mL of potassium oxybate, 0.096 g/mL of magnesium oxybate, and 0.040 g/mL of sodium oxybate (which contains 0.413 g/mL of GHB).

In some embodiments, the formulation is suitable for administration in a single or multiple dosage regimen per day. See U.S. Ser. Nos. 16/688,797, 62/769,380 and 62/769,382.

Any of the above formulations may be prepared and/or packaged as a powdered or dry form for mixing with an aqueous medium before oral administration, or they may be prepared in an aqueous medium and packaged. After mixing with an aqueous medium, preferably to prepare a solution, these formulations are resistant to both microbial growth and chemical conversion of GHB to GBL, thereby increasing the shelf-life of therapeutic formulations of GHB in an aqueous medium. These formulations then provide an easily titratable liquid medium for measuring the dosage of GHB to be administered to a patient.

The GHB may be lyophilized for more ready formulation into a desired vehicle or medium where appropriate. The active compounds may be formulated for parenteral administration, e.g., formulated for injection via intravenous, intraarterial, intramuscular, sub-cutaneous, intralesional, intraperitoneal or other parenteral routes. The preparation of a composition that comprises an aqueous solution that contains a GHB agent as an active component or ingredient will be known to those of skill in the art in light of the present disclosure. Typically, such compositions can be prepared as injectables, either as liquid solutions or suspensions. Solid forms suitable for using to prepare solutions or suspensions upon the addition of a liquid prior to injection can also be prepared; and the preparations can also be emulsified. See U.S. Pat. Nos. 6,472,431; 6,780,889; 7,262,219; 8,263,650; 8,461,203, 8,591,922, 8,901,173, 9,132,107, 9,555,017, 9,795,567, 10,195,168, U.S. Ser. Nos. 16/688,797, 62/769,380 and 62/769,382, and U.S. Patent Publication Nos. 2018/0263936 and 2012/0076865 for example for more information about parenteral administration.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For oral therapeutic administration, the active compounds may be incorporated with excipients and used in the form of beads, pellets, granules, tablets, buccal tablets or tabs, troches, capsules, elixirs, suspensions, syrups, wafers, and the like, to be admixed with an aqueous medium. Such compositions and preparations should contain at least 0.1% of active compound. The percentage of the compositions and preparations may, of course, be varied and may conveniently be between about 2-75% of the weight of the unit, or preferably between 25-60%. The amount of active compounds in such therapeutically useful compositions is such that a suitable dosage will be obtained. See U.S. Pat. Nos. 6,472,431; 6,780,889; 7,262,219; 8,263,650; 8,461,203, 8,591,922, 8,901,173, 9,132,107, 9,555,017, 9,795,567, 10,195,168, U.S. Ser. Nos. 16/688,797, 62/769,380 and 62/769,382, and U.S. Patent Publication No. 2018/0263936 and 2012/0076865 for example.

In some embodiments, the oxybate or a pharmaceutically acceptable salt thereof is administered when the patient wants to go to sleep. In some embodiments, the oxybate or a pharmaceutically acceptable salt thereof is administered at bedtime and about 2.5 h-4 h after the bedtime administration. In some embodiments, the oxybate or a pharmaceutically acceptable salt thereof is administered after a period of sleep.

### Methods of the Present Disclosure

The present disclosure provides, in part, methods for treating patient subpopulations (such a fibromyalgia, PTSD, IBS or IBD subpopulation) that, based on their % slow wave sleep, are likely (or have a higher probability) to respond to oxybate therapy. More particularly, it has been observed that a deficit in slow wave sleep prior to treatment is associated with a differentially greater response to oxybate therapy. In some embodiments, patient subpopulations with a slow wave sleep deficit have an increased likelihood of responding favorably to oxybate therapy, compared to patient subpopulations who are not classified as having a slow wave sleep deficit prior to treatment. In some embodiments, patient subpopulations who exhibit a slow wave sleep deficit prior to treatment have a greater chance of a successful treatment with oxybate therapy, or have a larger magnitude of drug effect compared to patient subpopulations who are not classified as having a slow wave sleep deficit prior to treatment.

In some embodiments, the methods comprise: (a) identifying a patient subpopulation (such a fibromyalgia, PTSD, IBS or IBD subpopulation) with a slow wave sleep deficit as determined by polysomnography or other methods known in the art or described herein that can detect slow wave sleep, such as actigraphy, and peripheral arterial tone; and (b) administering a therapeutically effective amount of oxybate or a pharmaceutically acceptable salt thereof to the patient. In some embodiments, the identified patient exhibits SWS of less than about 15%. In some embodiments, the identified patient exhibits SWS of less than about 10%. In some embodiments, the identified patient exhibits SWS of less than about 5%. In some embodiments, the identified patient exhibits SWS of less than about 1%.

Methods of Treating Fibromyalgia

In one aspect, the present disclosure provides methods of treating fibromyalgia. In some embodiments, the present disclosure provides a method of treating fibromyalgia, the method comprising: (a) identifying a fibromyalgia patient with a slow wave sleep (SWS) deficit as determined by polysomnography or other methods known in the art or described herein that can detect slow wave sleep, such as actigraphy, and peripheral arterial tone; and (b) administering a therapeutically effective amount of oxybate or a pharmaceutically acceptable salt thereof to the patient. In some embodiments, the present disclosure provides a method of treating fibromyalgia, the method comprising: (a) identifying a fibromyalgia patient with a slow wave sleep (SWS) deficit as determined by polysomnography; and (b) administering a therapeutically effective amount of oxybate or a pharmaceutically acceptable salt thereof to the patient. In some embodiments, the identified patient exhibits SWS of less than about 15%. In some embodiments, the identified patient exhibits SWS of less than about 10%. In some embodiments, the identified patient exhibits SWS of less than about 5%. In some embodiments, the identified patient exhibits SWS of less than about 1%. See U.S. Pat. No. 5,990,162.

In some embodiments, a fibromyalgia a patient with a slow wave sleep deficit (e.g., less than about 15% SWS, less than about 10% SWS, less than about 5% SWS, or less than about 1% SWS) as determined by polysomnography (e.g., electroencephalogram (EEG)) is more likely to respond to oxybate treatment than a patient who is not classified as having a SWS deficit.

In some embodiments, a fibromyalgia a patient with a slow sleep deficit and no psychiatric medical history is more likely to respond to oxybate treatment than a patient who is not classified as having a SWS deficit and has a psychiatric medical history.

In some embodiments, a fibromyalgia a patient with a slow sleep deficit and NREM 2 of % is more likely to respond to oxybate treatment than a patient who is not classified as having a SWS deficit and has a psychiatric medical history.

Methods of diagnosing a patient with fibromyalgia are known to those skilled in the art. In some embodiments, a patient is diagnosed with fibromyalgia using a validated clinical test. In some embodiments, a patient is diagnosed with fibromyalgia using criteria set forth in the American College of Rheumatology (ACR) criteria for fibromyalgia (Wolfe et al 1990).

In some embodiments, the efficacious dose (or therapeutically effective dose) is a dose that improves at least one symptom of the patient's fibromyalgia as measured by an improvement in the patient's Fibromyalgia Impact Questionnaire (FIQ) Score, Pain Visual Analogue Scale (P-VAS), Fatigue Visual Analogue Scale (F-VAS) score, Tender Points Index (TPI) score, or Tender Points Count (TPC) score compared to baseline prior to the treatment. In some embodiments, the efficacious dose (or therapeutically effective dose) is a dose that provides greater than the minimum clinically important difference in at least one symptom of the patient's Fibromyalgia, as measured by an improvement in the patient's Impact Questionnaire (FIQ) Score, Pain Visual Analogue Scale (P-VAS), Fatigue Visual Analogue Scale (F-VAS) score, Tender Points Index (TPI) score, or Tender Points Count (TPC) score compared to baseline prior to the treatment. In some embodiments, therapeutically effective dose is a dose that increases the patient's percentage of SWS compared to baseline prior to the treatment. In some embodiments, the therapeutically effective dose is a dose that increases the patient's percentage of REM sleep compared to baseline prior to the treatment. In some embodiments, the therapeutically effective dose is a dose that provides improved sleep quality as determined by the Pittsburgh Sleep Quality Index (PSQI) compared to baseline prior to the treatment.

In some embodiments, the administration of the oxybate or a pharmaceutically acceptable salt thereof provides at least about 15%, at least about 20%, at least about 30%, or at least bout 40%, improvement in Pain Visual Analogue Scale (P-VAS) score or Fatigue Visual Analogue Scale (F-VAS) score compared to baseline prior to treatment in a patient identified as having a slow wave sleep deficit. In some embodiments, the administration of the oxybate or a pharmaceutically acceptable salt thereof provides at least about 30% improvement in Pain Visual Analogue Scale (P-VAS) score compared to baseline prior to the treatment in a patient identified as having a slow wave sleep deficit. In some embodiments, the administration of the oxybate or a pharmaceutically acceptable salt thereof provides at least about 30% improvement in Fatigue Visual Analogue Scale (F-VAS) score compared to baseline prior to the treatment, in a patient identified as having a slow wave sleep deficit. In some embodiments, the identified patient exhibits SWS of less than about 15%. In some embodiments, the identified patient exhibits SWS of less than about 10%. In some embodiments, the identified patient exhibits SWS of less than about 5%. In some embodiments, the identified patient exhibits SWS of less than about 1%. In some embodiments, the administration of the oxybate or a pharmaceutically acceptable salt thereof provides at least about 30% improvement in Pain Visual Analogue Scale (P-VAS) score or Fatigue Visual Analogue Scale (F-VAS) score compared to baseline prior to the treatment in a patient identified as having a less than about 10% of slow wave sleep. In some embodiments, some embodiments, the administration of the oxybate or a pharmaceutically acceptable salt thereof provides at least about 30% improvement in Pain Visual Analogue Scale (P-VAS) score or Fatigue Visual Analogue Scale (F-VAS) score compared to baseline prior to the treatment in a patient identified as having a less than about 5% of slow wave sleep. In some embodiments, the administration of the oxybate or a pharmaceutically acceptable salt thereof provides a MCID in the patients FIQ score of about 10%, about 12%, about 14%, about 16%, about 18%, or about 20%. In some embodiments, the administration of the oxybate or a pharmaceutically acceptable salt thereof provides a MCID in the patients FIQ score in the FIQ score of about 14%.

Methods of Treating PTSD

In one aspect, the present disclosure provides methods of treating PTSD. In some embodiments, the present disclosure provides a method of treating post-traumatic stress disorder (PTSD) in a patient in need thereof, the method comprising: (a) identifying a patient with PTSD with a SWS deficit as determined by polysomnography or other methods known in the art or described herein that can detect slow wave sleep, such as actigraphy, and peripheral arterial tone; and (b) administering a therapeutically effective amount of oxybate or a pharmaceutically acceptable salt thereof. In some embodiments, the present disclosure provides a method of treating post-traumatic stress disorder (PTSD) in a patient in need thereof, the method comprising: (a) identifying a patient with PTSD with a SWS deficit as determined by polysomnography; and (b) administering a therapeutically effective amount of oxybate or a pharmaceutically acceptable salt thereof. In some embodiments, the oxybate or a pharmaceutically acceptable salt thereof is used to treat a patient with PTSD, to treat PTSD, or to treat the symptoms of PTSD. In some embodiments, the identified patient exhibits SWS of less than about 15%. In some embodiments, the identified patient exhibits SWS of less than about 10%. In some embodiments, the identified patient exhibits SWS of less than about 5%. In some embodiments, the identified patient exhibits SWS of less than about 1%.

In some embodiments, a PTSD patient with a slow wave sleep deficit (e.g. less than about 15%, less than about 10%, less than about 5%, or less than about 1%) as determined by polysomnography (e.g. sleep electroencephalogram (EEG)) is likely to respond to treatment with a therapeutically effective amount of oxybate or a pharmaceutically acceptable salt.

Methods of diagnosing a patient with PTSD are known to those skilled in the art. In some embodiments, a patient is diagnosed with PTSD using a validated clinical test. In some embodiments, a patient is diagnosed with PTSD using the criteria set forth in Diagnostic and Statistical Manual of Mental Disorders (DSM-5; 1). In some embodiments, a patient is diagnosed with PTSD using the criteria set forth in a prior version of the Diagnostic and Statistical Manual of Mental Disorders. In some embodiments, a patient is diagnosed with PTSD using Clinician-Administered PTSD Scale for DSM-5 (CAPS-5). In some embodiments, a patient is diagnosed with PTSD using the criteria set forth in a prior version of using Clinician-Administered PTSD Scale for DSM. In some embodiments, PTSD severity is evaluated by CAPS-5. In some embodiments, a patient is diagnosed with PTSD using the Posttraumatic Stress Disorder Checklist for DSM-5 (PCL-5) criteria. In some embodiments, a patient is diagnosed with PTSD using a validated clinical test to diagnose PTSD.

In some embodiments, the efficacious dose (or therapeutically effective dose) is a dose that improves at least one symptom of the patient's PTSD as measured by an improvement in the patient's CAPS-5 score or the patient's PCL-5 score compared to baseline prior to the treatment. In some embodiments, the efficacious dose (or therapeutically effective dose) is a dose that provides greater than the minimum clinically important difference in at least one symptom of the patient's PTSD, as measured by an improvement in the patient's CAPS-5 score or PCL-5 score compared to baseline prior to the treatment. In some embodiments, the efficacious dose (or therapeutically effective dose) is a dose that provides a reduction in the patients total CAPS-5 score of about or at least about 7 points, about or at least about 8 points, about or at least about 9 points, about or at least about 10 points, about or at least about 11 points, about or at least about 12 points, about or at least about 13 points, or about or at least about 15 points. In some embodiments, the efficacious dose (or therapeutically effective dose) is a dose that provides a reduction in the patients total PCL-5 score of about or at least about 5 points, about or at least about 6 points, about or at least about 7 points, about or at least about 8 points, about or at least about 9 points, or about or at least about 10 points. In some embodiments, therapeutically effective dose is a dose that increases the patient's percentage of SWS compared to baseline prior to the treatment. In some embodiments, the therapeutically effective dose is a dose that increases and/or consolidates the patient's percentage of REM sleep compared to baseline prior to the treatment. In some embodiments, the therapeutically effective dose is a dose that provides improved sleep quality as determined by a patient-centric measurement (e.g., the Pittsburgh Sleep Quality Index (PSQI)) compared to baseline prior to the treatment.

In some embodiments, the administration of the oxybate or a pharmaceutically acceptable salt thereof provides an improvement in the patient's sleep-related PTSD symptoms. For example, the administration of the oxybate or a pharmaceutically acceptable salt thereof provides an improvement in the patient's insomnia, nightmares, or somniphobia, associated with PTSD compared to prior to the treatment. In some embodiments, the administration of the oxybate or a pharmaceutically acceptable salt thereof provides increased percentage of SWS compared to prior to the administration. In some embodiments, the administration of the oxybate or a pharmaceutically acceptable salt thereof provides increased percentage of REM sleep compared to prior to the administration. In some embodiments, the administration of the oxybate or a pharmaceutically acceptable salt thereof provides improved sleep quality as determined by a patient-centric measurement (e.g., the Pittsburgh Sleep Quality Index (PSQI)) compared to baseline prior to the treatment.

Methods of Treating IBS and IBD

In one aspect, the present disclosure provides methods of treating IBS or IBD. In some embodiments, the present disclosure provides methods of treating IBS in a patient in need thereof, the method comprising: (a) identifying a patient with IBS with a SWS deficit as determined by polysomnography or other methods known in the art or described herein that can detect slow wave sleep, such as actigraphy, and peripheral arterial tone; and (b) administering a therapeutically effective amount of oxybate or a pharmaceutically acceptable salt thereof. In some embodiments, the present disclosure provides methods of treating IBS in a patient in need thereof, the method comprising: (a) identifying a patient with IBS with a slow wave sleep deficit as determined by polysomnography; and (b) administering a therapeutically effective amount of oxybate or a pharmaceutically acceptable salt thereof. In some embodiments, the oxybate or a pharmaceutically acceptable salt thereof is used to treat a patient with IBS, to treat IBS, or to treat the symptoms of IBS. In some embodiments, the identified patient exhibits SWS of less than about 15%. In some embodiments, the identified patient exhibits SWS of less than about 10%. In some embodiments, the identified patient exhibits SWS of less than about 5%. In some embodiments, the identified patient exhibits SWS of less than about 1%.

In some embodiments, an IBS patient with a slow wave sleep deficit (e.g. less than about 15%, less than about 10%, less than about 5%, or less than about 1%) as determined by polysomnography (e.g. sleep electroencephalogram (EEG)) is likely to respond to treatment with a therapeutically effective amount of oxybate or a pharmaceutically acceptable salt.

Methods of diagnosing a patient with IBS are known to those skilled in the art. In some embodiments, a patient is diagnosed with IBS using a validated clinical test. In some embodiments, a patient is diagnosed with IBS using the criteria set forth in the Rome IV Diagnostic Criteria for Irritable Bowel Syndrome (IBS). According to the Rome IV Diagnostic Criteria for IBS, a patient with IBS has recurrent abdominal pain, on average, at least 1 day per week in the last three months, associated with two or more of: (1) related to defecation, (2) associated with a change in the frequency of stool; (3) associated with a change in form (appearance) of stool. The criteria should be fulfilled for the last three months with symptom onset at least six months prior to diagnosis. In some embodiments, the patient with IBS has IBS with constipation (IBS-C), IBS with diarrhea (IBS-D), or mixed IBS (IBS-M). In some embodiments, a patient is diagnosed with IBS using the criteria set forth in a prior version of the Rome IV Diagnostic Criteria for Irritable Bowel Syndrome (IBS).

In some embodiments, the efficacious dose (or therapeutically effective dose) is a dose that improves at least one symptom of the patient's IBS as measured by an improvement in the patient's IBS Global Improvement scale score compared to baseline prior to the treatment. In some embodiments, the efficacious dose (or therapeutically effective dose) is a dose that provides greater than the minimum clinically important difference in at least one symptom of the patient's IBS, as measured by an improvement in the patient's IBS Global Improvement scale score compared to baseline prior to the treatment. In some embodiments, therapeutically effective dose is a dose that increases the patient's percentage of SWS compared to baseline prior to the treatment. In some embodiments, the therapeutically effective dose is a dose that increases the patient's percentage of REM sleep compared to baseline prior to the treatment. In some embodiments, the therapeutically effective dose is a dose that provides improved sleep quality as determined by the Pittsburgh Sleep Quality Index (PSQI) compared to baseline prior to the treatment.

In some embodiments, the administration of the oxybate or a pharmaceutically acceptable salt thereof provides an improvement in the patient's sleep symptoms associated with IBS compared to prior to the treatment. For example, the administration of the oxybate or a pharmaceutically acceptable salt thereof provides an improvement in the patient's insomnia associated with IBS compared to prior to the treatment. In some embodiments, the administration of the oxybate or a pharmaceutically acceptable salt thereof provides increased percentage of SWS compared to prior to the administration. In some embodiments, the administration of the oxybate or a pharmaceutically acceptable salt thereof provides increased percentage of REM sleep compared to prior to the administration. In some embodiments, the administration of the oxybate or a pharmaceutically acceptable salt thereof provides improved sleep quality as determined by the Pittsburgh Sleep Quality Index (PSQI).

The present disclosure also provides, in part, methods of treating IBD in a patient in need thereof, the method comprising: (a) identifying a patient with IBD with a SWS deficit as determined by polysomnography (e.g., by sleep EEG), or other methods known in the art or described herein that can detect slow wave sleep, such as actigraphy, and peripheral arterial tone; and (b) administering a therapeutically effective amount of oxybate or a pharmaceutically acceptable salt thereof. In some embodiments, the present disclosure also provides, in part, methods of treating IBD in a patient in need thereof, the method comprising: (a) identifying a patient with IBD with a slow wave sleep deficit as determined by polysomnography (e.g., by sleep EEG); and (b) administering a therapeutically effective amount of oxybate or a pharmaceutically acceptable salt thereof. In some embodiments, the oxybate or a pharmaceutically acceptable salt thereof is used to treat a patient with IBD, to treat IBD, or to treat the symptoms of IBD. In some embodiments, the identified patient exhibits SWS of less than about 15%. In some embodiments, the identified patient exhibits SWS of less than about 10%. In some embodiments, the identified patient exhibits SWS of less than about 5%. In some embodiments, the identified patient exhibits SWS of less than about 1%.

In some embodiments, an IBD patient with a slow wave sleep deficit (e.g. less than about 15%, less than about 10%, less than about 5%, or less than about 1%) as determined by polysomnography (e.g. sleep electroencephalogram (EEG)) is likely to respond to treatment with a therapeutically effective amount of oxybate or a pharmaceutically acceptable salt.

Methods of diagnosing a patient with IBD are known to those skilled in the art. In some embodiments, a patient is diagnosed with IBD using a validated clinical test. In some embodiments, a patient is diagnosed with IBD using a blood test, an x-ray, a colonoscopy, an endoscopy, or leukocyte scintigraphy. In some embodiments, leukocyte scintigraphy determines whether white blood cells tagged with a radioactive substance migrate to the gastrointestinal (GI) tract.

In some embodiments, the efficacious dose (or therapeutically effective dose) is a dose that improves at least one symptom of the patient's IBD. For example, in some embodiments, the efficacious dose (or therapeutically effective dose) is a dose that provides an improvement in clinical response, patient reported outcomes (e.g., including e.g., inflammatory Bowel Disease Questionnaire score, the Manitoba IBD Index score, the numeric rating scale score, and the IBD-Control questionnaire score), quality of life, disability, endoscopic or histology assessment scales, underlying inflammatory activity, structural damage, fatigue, biomarker endpoints, safety endpoints, or combinations thereof. In some embodiments, the therapeutically effective dose is a dose that increases the patient's percentage of REM sleep compared to baseline prior to the treatment. In some embodiments, the therapeutically effective dose is a dose that provides improved sleep quality as determined by the Pittsburgh Sleep Quality Index (PSQI) compared to baseline prior to the treatment.

In a patient with Crohn's disease the efficacious dose (or therapeutically effective dose) is a dose that improves at least one symptom of the patient's Crohn's disease as measured by an improvement in the patients Crohn's Disease Activity Index (CDAI) score compared to baseline. In some embodiments, the efficacious dose (or therapeutically effective dose) is a dose that provides greater than the minimum clinically important difference in at least one symptom of the patient's Crohn's disease, as measured by an improvement in the patient's CDAI score compared to baseline prior to the treatment. In some embodiments, oxybate therapy results in a CDAI score of less than about 150. In some embodiments, the efficacious dose is a dose that improves at least one symptom of the patient's Crohn's disease as measured by an improvement in the patient's PRO-2 score, Harvey Bradshaw Index (HBI) score, van Hees index score, Physician Global Assessment score, or Perianal Disease Activity Index (PDAI) score.

In a patient with ulcerative colitis, the efficacious dose (or therapeutically effective dose) is a dose that improves at least one symptom of the patient's ulcerative colitis. For example, an improvement in at least one symptom as measured by an improvement in the patients Mayo score or UC Disease Activity Index (UCDAI) score compared to baseline. In some embodiments, the efficacious dose (or therapeutically effective dose) is a dose that provides greater than the minimum clinically important difference in at least one symptom of the patient's ulcerative colitis, as measured by an improvement in the patient's Mayo score or UCDAI score compared to baseline prior to the treatment. In some embodiments, the efficacious dose is a dose that improves at least one symptom of the patients ulcerative colitis as measured by an improvement in the patients Rachmilewitz Score, Walmsley score, Lichtiger Index score, Seo Index score or Powell-Tuck Index score compared to baseline.

In some embodiments, the administration of the oxybate or a pharmaceutically acceptable salt thereof provides an improvement in the patient's sleep symptoms associated with IBD compared to prior to the treatment. For example, the administration of the oxybate or a pharmaceutically acceptable salt thereof provides an improvement in the patient's insomnia associated with IBD compared to prior to the treatment. In some embodiments, the administration of the oxybate or a pharmaceutically acceptable salt thereof provides increased percentage of SWS compared to prior to the administration. In some embodiments, the administration of the oxybate or a pharmaceutically acceptable salt thereof provides increased percentage of REM sleep compared to prior to the administration. In some embodiments, the administration of the oxybate or a pharmaceutically acceptable salt thereof provides improved sleep quality as determined by the Pittsburgh Sleep Quality Index (PSQI).

The methods of the present disclosure comprise the step of identifying a patient with a slow wave sleep deficit. As used herein, a slow wave sleep deficit means less slow wave sleep than is normal based on the patient's age and/or biological sex.

As used in the methods of the present disclosure, the following are considered normal % SWS for males by age group:

| Age (years) | Normal % SWS |
|---|---|
| 20-29 | About 14.5% |
| 30-40 | About 9.0% |
| 37-53 | About 11% |
| 54-60 | About 8% |
| 61 to 70 | About 7% |
| ≥71 | about 5.5% |

As used in the methods of the present disclosure, the following are considered normal % SWS for females by age group:

| Age (years) | Normal % SWS |
|---|---|
| 20-29 | About 15.5% |
| 30-40 | About 13% |
| 37-53 | About 14% |
| 54-60 | About 17% |
| 61 to 70 | About 17% |
| ≥71 | About 17% |

Thus, if a patient is expected to about 17% SWS based on their age and/or biological sex (for example, females ages 61 to 70), then a patient exhibits a SWS deficit if their SWS is less than about 17%, including about 0%, about 1%, about 2%, about 3%, about 4%, about 5%, about 6%, about 7%, about 8%, about 9%, about 10%, about 11%, about 12%, about 13%, about 14%, about 15%, or about 16%, including any subrange and value therebetween.

In some embodiments, a SWS deficit is about 1% to about 95% of the normal SWS based on the patient's age and/or biological sex, including about 1%, about 5%, about 10%, about 15%, about 20%, about 25%, about 30%, about 40%, about 50%, about 60%, about 70%, about 80%, about 90%, to about 95%, or any subrange and value therebetween.

The methods of the present disclosure comprise identifying a patient with a slow wave sleep deficit (SWS) as determined by polysomnography (e.g. sleep electroencephalogram (EEG)). In some embodiments, the identified patient exhibits SWS of less than about 20%, less than about 19%, less than about 18%, less than about 17%, less than about 16%, less than about 15%, less than about 14%, less than about 13%, less than about 12%, less than about 11%, less than about 10%, less than about 9.5%, less than about 9%, less than about 8.5%, less than about 8%, less than about 7.5%, less than about 7%, less than about 6.5%, less than about 6%, less than about 5.5%, less than about less than about 5%, less than about 4.5%, less than about 4%, less than about 3.5%, less than about 3%, less than about 2.5%, less than about 2%, less than about 1.5%, less than about 1%, or less than about 0.5%. In some embodiments, the identified patient exhibits no slow wave sleep. In some embodiments, the identified patient exhibits SWS of less than about 10%. In some embodiments, the identified patient exhibits SWS of less than about 5%. In some embodiments, the identified patient exhibits SWS in the range of 0% to about 20%. In some embodiments, the identified patient exhibits SWS in the range of about 0% to about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, to about 20%, including any sub-range therebetween.

In some embodiments, the identified patient exhibits less than about 15 min stage 3/4 sleep as determined by polysomnography (e.g. sleep electroencephalogram (EEG)). In some embodiments, the identified patient exhibits less than about 14 min stage 3/4 sleep as determined by polysomnography (e.g. sleep electroencephalogram (EEG)). In some embodiments, the identified patient exhibits less than about 13.5 min at stage 3/4 sleep as determined by polysomnography (e.g. sleep electroencephalogram (EEG)).

In some embodiments, the oxybate or a pharmaceutically acceptable salt thereof that is administered comprises sodium oxybate.

In some embodiments, the oxybate or a pharmaceutically acceptable salt thereof that is administered may be any of the mixed salt oxybate compositions described herein (e.g., JZP-258). In some embodiments, the relative amount of each salt in the mixed salt oxybate that is administered is expressed in terms of wt/wt %. In some embodiments, the mixed salt oxybate comprises sodium oxybate, potassium oxybate, magnesium oxybate and calcium oxybate, and wherein the mixed salt oxybate comprises about 5%-40% of sodium oxybate (wt/wt %). In some embodiments, the mixed salt oxybate comprises about 5%-40% of sodium oxybate (wt/wt %), about 10%-40% of potassium oxybate (wt/wt %), about 5%-30% of magnesium oxybate (wt/wt %), and about 20%-80% of calcium oxybate (wt/wt %). In some embodiments, the mixed salt oxybate comprises about 8% of sodium oxybate (wt/wt %), about 25.5% of potassium oxybate (wt/wt %), about 19.5% of magnesium oxybate (wt/wt %) and about 47% of calcium oxybate (wt/wt %).

In some embodiments, the relative amount of each salt in the mixed salt oxybate (e.g., JZP-258) that is administered in a liquid pharmaceutical composition is expressed in terms of wt/vol %. In some embodiments, the liquid pharmaceutical composition comprises a mixed salt oxybate comprising sodium oxybate, potassium oxybate, magnesium oxybate and calcium oxybate, and wherein the mixed salt oxybate comprises about 5%-40% of sodium oxybate (wt/vol %). In some embodiments, the liquid pharmaceutical composition comprises a mixed salt oxybate comprising about 5%-40% of sodium oxybate (wt/vol %), about 10%-40% of potassium oxybate (wt/vol %), about 5%-30% of magnesium oxybate (wt/vol %), and about 20%-80% of calcium oxybate (wt/vol %). In some embodiments, the liquid pharmaceutical composition comprises the mixed salt oxybate comprising about 8% of sodium oxybate (wt/vol %), about 26% of potassium oxybate (wt/vol %), about 19.2% of magnesium oxybate (wt/vol %) and about 46.8% of calcium oxybate (wt/vol %).

In some embodiment, the mixed salt oxybate comprises about 8% mol. equiv. of sodium oxybate, about 23% mol. equiv. of potassium oxybate, about 21% mol. equiv. of magnesium oxybate and about 48% mol. equiv. calcium oxybate.

In some embodiments, the dose of the oxybate or a pharmaceutically acceptable salt thereof is described in terms of the amount of the oxybate or a pharmaceutically acceptable salt thereof that is administered to the patient. In some embodiments, about 0.25 g-12.0 g of the oxybate or a pharmaceutically acceptable salt thereof is administered per day. In some embodiments, about 3 g-12.0 g of the oxybate or a pharmaceutically acceptable salt thereof is administered per day. In some embodiments, about 0.25 g-10.0 g of the oxybate or a pharmaceutically acceptable salt thereof is administered per day. In some embodiments, about 2.0 g-10.0 g of the oxybate or a pharmaceutically acceptable salt thereof is administered per day. In some embodiments, about 3.0 g-9.5 g of the oxybate or a pharmaceutically acceptable salt thereof is administered per day. In some embodiments, about 4.5 g-9.0 g of the oxybate or a pharmaceutically acceptable salt thereof is administered per day. In some embodiments, about 3 g-9 g of the oxybate of a pharmaceutically acceptable salt thereof is administered per day. In some embodiments, about 6 g-9 g of the oxybate of a pharmaceutically acceptable salt thereof is administered per day. In some embodiments, about 4.5 g-6 g of the oxybate of a pharmaceutically acceptable salt thereof is administered per day. In some embodiments, about 0.5 g, about 1.0 g, about 1.5 g, about 2.0 g, about 2.5 g, about 3.0 g, about 3.5, about 4.0 g, about 4.5 g, about 5.0 g, about 5.5 g, about 6.0 g, about 6.5 g, about 7.0 g, about 7.5 g, about 8.0 g, about 8.5 g, about 9.0 g, about 9.5 g, about 10.0 g, about 10.5 g, about 11.0 g, about 11.5 g, or about 12 g of the oxybate or a pharmaceutically acceptable salt thereof is administered per day. In some embodiments, about 4.5 g of the oxybate or a pharmaceutically acceptable salt thereof is administered per day. In some embodiments, about 6.0 g of the oxybate or a pharmaceutically acceptable salt thereof is administered per day. In some embodiments, about 7.5 g of the oxybate or a pharmaceutically acceptable salt thereof is administered per day. In some embodiments, about 9.0 g of the oxybate or a pharmaceutically acceptable salt thereof is administered per day. A daily dose can be administered in a single or divided (equally or unequally) dose. In some embodiments, the oxybate or a pharmaceutically acceptable salt thereof is administered three times per day. In some embodiments, the oxybate or a pharmaceutically acceptable salt thereof is administered twice per day. In some embodiments, the oxybate or a pharmaceutically acceptable salt thereof is administered once per day, See published U.S. Patent Publication No. 2020/0330393. In some embodiments, the oxybate or a pharmaceutically acceptable salt thereof is administered at bedtime. In some embodiments, the oxybate or a pharmaceutically acceptable salt thereof is administered at bedtime and about 2.5 h-4 h after the bedtime administration. In some embodiments, the oxybate or a pharmaceutically acceptable salt thereof is administered after a period of sleep. In some embodiments, the oxybate or a pharmaceutically acceptable salt thereof is administered after a period of sleep and about 2.5 h-4 h later.

In some embodiments, the oxybate or a pharmaceutically acceptable salt thereof is administered once per day (e.g., nightly). For example, in some embodiments, about 1.0 g, about 1.5 g, about 2.0 g, about 2.5 g, about 3.0 g, about 3.5 g, about 4.0 g, about 4.5 g, about 5.0 g, about g, about 6.0 g, about 6.5 g, about 7.0 g, about 7.5 g, about 8.0 g, about 8.5 g, about 9.0 g, about 9.5 g, about 10.0 g, about 10.5 g, about 11.0 g, about 11.5 g, or about 12.0 g of the oxybate or a pharmaceutically acceptable salt thereof is administered once per day.

In some embodiments, the oxybate or a pharmaceutically acceptable salt thereof is administered twice per day. For example, in some embodiments, about 0.25 g, about 0.5 g, about g, about 1.0 g, about 1.25 g, about 1.5 g, about 1.75 g, about 2.0 g, about 2.25 g, about 2.5 g, about 2.75 g, about 3.0 g, about 3.25 g, about 3.5 g, about 3.75 g, about 4.0 g, about 4.25, about 4.5 g, about 4.75 g, about 5.00 g, about 5.25 g, about 5.50 g, about 5.75 g or, about 6.00 g of the oxybate or a pharmaceutically acceptable salt thereof is administered twice per day. In some embodiments, about 2.25 g of the oxybate or a pharmaceutically acceptable salt thereof is administered twice per day. In some embodiments, about 3.0 g of the oxybate or a pharmaceutically acceptable salt thereof is administered twice per day. In some embodiments, about 3.75 g of the oxybate or a pharmaceutically acceptable salt thereof is administered twice per day. In some embodiments, about 4.5 g of the oxybate or a pharmaceutically acceptable salt thereof is administered twice per day.

In some embodiments, the oxybate or a pharmaceutically acceptable salt thereof is administered three times per day. For example, in some embodiments, about 0.17 g, about 0.33 g, about 0.50 g, about 0.66 g, about 0.83 g, about 1.0 g, about 1.16 g, about 1.32 g, about 1.5 g, about 1.67 g, about 1.83 g, about 2.0 g, about 2.3 g, about 2.5 g, about 2.67 g, about 2.83 g, about 3.0 g, about 3.17 g, about 3.33 g, about 3.5 g, about 3.67 g, about 3.83 g, or about 4.0 g of the oxybate or a pharmaceutically acceptable salt thereof is administered three times per day.

In some embodiments, the methods of the present disclosure comprise:
(a) administering an initial daily dose of the oxybate or a pharmaceutically acceptable salt thereof to the patient and
(b) titrating the dose to provide a therapeutically effective amount of the oxybate or a pharmaceutically acceptable salt thereof.

In some embodiments, a therapeutically effective dose is achieved by starting the patient on an initial daily dose and titrating to an efficacious and tolerated dose by gradually increasing or decreasing the daily administered amount of mixed salt oxybate until a dose that is effective (e.g., the patient with fibromyalgia, PTSD, IBD or IBS is treated) and tolerated is achieved.

In some embodiments, after a dose that is effective and tolerated is achieved the administered dose is further adjusted to optimize the patient's treatment. The dose may be optimized by the methods described herein (for example, up-titrating or down-titrating the dose, change the number of daily administrations, the patient's dose up or changing the division of the total nightly dosing).

In some embodiments, the initial daily dose is from about 0.5 g to about 4.5 g of the oxybate or a pharmaceutically acceptable salt thereof. In some embodiments, the initial daily dose is less than about 4.5 g of the oxybate or a pharmaceutically acceptable salt thereof. In some embodiments, the initial daily dose is about 0.25 g of the oxybate or a pharmaceutically acceptable salt thereof. In some embodiments, the initial daily dose is about 0.50 g of the oxybate or a pharmaceutically acceptable salt thereof. In some embodiments, the initial daily dose is about 1.0 g of the oxybate or a pharmaceutically acceptable salt thereof. In some embodiments, the initial daily dose is about 1.5 g of the oxybate or a pharmaceutically acceptable salt thereof. In some embodiments, the initial daily dose is about 2.0 g of the oxybate or a pharmaceutically acceptable salt thereof. In some embodiments, the initial daily dose is about 4.5 g of the oxybate or a pharmaceutically acceptable salt thereof.

In some embodiments, the titration comprises administering ascending doses of the oxybate or a pharmaceutically acceptable salt thereof. In some embodiments, the daily dose is increased every week until a dose that is effective and tolerated is achieved. In some embodiments, the daily dose is increased every other week until a dose that is effective and tolerated is achieved. In some embodiments, the daily dose is increased every third week until a dose that is effective and tolerated is achieved.

In some embodiments, the daily dose is increased on a weekly basis until a dose that is effective and tolerated is achieved. In some embodiments, the daily dose is increased once every week until a dose that is effective and tolerated is achieved. In some embodiments, the daily dose is increased twice every week until a dose that is effective and tolerated is achieved. In some embodiments, the daily dose is increased three times every week until a dose that is effective and tolerated is achieved. In some embodiments, the daily dose is increased four times every week until a dose that is effective and tolerated is achieved. In some embodiments, the daily dose is increased five times every week until a dose that is effective and tolerated is achieved. In some embodiments, the daily dose is increased six times every week until a dose that is effective and tolerated is achieved. In some embodiments, the total weekly dose is increased by less than about 1.5 g of the oxybate or a pharmaceutically acceptable salt thereof.

In some embodiments, the daily dose is increased every day until a dose that is effective and tolerated is achieved. In some embodiments, the daily dose is increased every other day until a dose that is effective and tolerated is achieved. In some embodiments, the daily dose is increased every second or third day until a dose that is effective and tolerated is achieved. In some embodiments, the daily dose is increased every third day until a dose that is effective and tolerated is achieved. In some embodiments, the daily dose is increased every second, third, or fourth day until a dose that is effective and tolerated is achieved. In some embodiments, the daily dose is increased every fourth day until a dose that is effective and tolerated is achieved. In some embodiments, the daily dose is increased every second, third, fourth, or fifth day until a dose that is effective and tolerated is achieved. In some embodiments, the daily dose is increased every fifth day until a dose that is effective and tolerated is achieved. In some embodiments, the daily dose is increased every second, third, fourth, fifth, or sixth day until a dose that is effective and tolerated is achieved. In some embodiments, the daily dose is increased every sixth day until a dose that is effective and tolerated is achieved. In some embodiments, the daily dose is increased every second, third, fourth, fifth, sixth, or seventh day until a dose that is effective and tolerated is achieved. In some embodiments, the daily dose is increased every seventh day until a dose that is effective and tolerated is achieved.

In some embodiments, the daily dose of the oxybate or a pharmaceutically acceptable salt thereof is increased by about 0.5 g to 1.5 g per week. In some embodiments, the daily dose is increased by about 0.25 g to 1.5 g per week. In some embodiments, the daily dose is increased by less than about 1.5 g per week.

In some embodiments, the titration comprises administering descending doses of the oxybate or a pharmaceutically acceptable salt thereof. In some embodiments, the daily dose is decreased every week until a dose that is effective and tolerated is achieved. In some embodiments, the daily dose is decreased every other week until a dose that is effective and tolerated is achieved. In some embodiments, the daily dose is decreased every third week until a dose that is effective and tolerated is achieved.

In some embodiments, the daily dose is decreased on a weekly basis until a dose that is effective and tolerated is achieved. In some embodiments, the daily dose is decreased once every week until a dose that is effective and tolerated is achieved. In some embodiments, the daily dose is decreased twice every week until a dose that is effective and tolerated is achieved. In some embodiments, the daily dose is decreased three times every week until a dose that is effective and tolerated is achieved. In some embodiments, the daily dose is decreased four times every week until a dose that is effective and tolerated is achieved. In some embodiments, the daily dose is decreased five times every week until a dose that is effective and tolerated is achieved. In some embodiments, the daily dose is decreased six times every week until a dose that is effective and tolerated is achieved.

In some embodiments, the daily dose is decreased every day until a dose that is effective and tolerated is achieved. In some embodiments, the daily dose is decreased every other day until a dose that is effective and tolerated is achieved. In some embodiments, the daily dose is decreased every second or third day until a dose that is effective and tolerated is achieved. In some embodiments, the daily dose is decreased every third day until a dose that is effective and tolerated is achieved. In some embodiments, the daily dose is decreased every second, third, or fourth day until a dose that is effective and tolerated is achieved. In some embodiments, the daily dose is decreased every fourth day until a dose that is effective and tolerated is achieved. In some embodiments, the daily dose is decreased every second, third, fourth, or fifth day until a dose that is effective and tolerated is achieved. In some embodiments, the daily dose is decreased every fifth day until a dose that is effective and tolerated is achieved. In some embodiments, the daily dose is decreased every second, third, fourth, fifth, or sixth day until a dose that is effective and tolerated is achieved. In some embodiments, the daily dose is decreased every sixth day until a dose that is effective and tolerated is achieved. In some embodiments, the daily dose is decreased every second, third, fourth, fifth, sixth, or seventh day until a dose that is effective and tolerated is achieved. In some embodiments, the daily dose is decreased every seventh day until a dose that is effective and tolerated is achieved.

In some embodiments, the daily dose is decreased by about 0.5 g to 1.5 g per week. In some embodiments, the daily dose is decreased by about 0.25 g to 1.5 g per week. In some embodiments, the daily dose is decreased by less than about 1.5 g per week. In some embodiments, the daily dose is decreased by about 0.5 g to 9.0 g per week. In some embodiments, the daily dose is decreased by about 0.25 g to 9.0 g per week.

In some embodiments, the titration step (b) comprises switching a patient from a once a day dose to a twice a day dose of the oxybate or a pharmaceutically acceptable salt thereof. In some embodiments, the titration step (b) comprises switching a patient from a twice a day dose to a three times a day dose of the oxybate or a pharmaceutically acceptable salt thereof. In some embodiments, the titration step (b) comprises switching a patient from a twice a day dose to a once a day dose of the oxybate or a pharmaceutically acceptable salt thereof. In some embodiments, the titration step (b) comprises switching a patient from a three times a day dose to a twice a day dose of the oxybate or a pharmaceutically acceptable salt thereof. In some embodiments, the titration step (b) is from about 1 week to about 14 weeks.

In some embodiments, the dose of the oxybate or a pharmaceutically acceptable salt thereof is described in terms of the amount of GHB that is administered to the patient. In some embodiments, the oxybate or a pharmaceutically acceptable salt thereof containing about 0.818 g-7.362 g, about 1.636 g-8.18 g; about 2.454 g-7.771 g; or about 3.681 g-7.362 g of GHB is administered per day.

In some embodiments, the oxybate or a pharmaceutically acceptable salt thereof containing about 0.818 g of GHB is administered per day. In some embodiments, the oxybate or a pharmaceutically acceptable salt thereof containing about 0.409 g of GHB is administered twice per day. In some embodiments, the oxybate or a pharmaceutically acceptable salt thereof containing about 0.273 g of GHB is administered three times per day.

In some embodiments, the oxybate or a pharmaceutically acceptable salt thereof containing about 2.454 g of GHB is administered per day. In some embodiments, the oxybate or a pharmaceutically acceptable salt thereof containing about 1.227 g of GHB is administered twice per day. In some embodiments, the oxybate or a pharmaceutically acceptable salt thereof containing about 0.818 g of GHB is administered three times per day.

In some embodiments, the oxybate or a pharmaceutically acceptable salt thereof containing about 3.681 g of GHB is administered per day. In some embodiments, the oxybate or a pharmaceutically acceptable salt thereof containing about 1.841 g of GHB is administered twice per day. In some embodiments, the oxybate or a pharmaceutically acceptable salt thereof containing about 1.227 g of GHB is administered three times per day.

In some embodiments, the oxybate or a pharmaceutically acceptable salt thereof containing about 4.908 g of GHB is administered per day. In some embodiments, the oxybate or a pharmaceutically acceptable salt thereof containing about 2.454 g of GHB is administered twice per day. In some embodiments, the oxybate or a pharmaceutically acceptable salt thereof containing about 1.636 g of GHB is administered three times per day.

In some embodiments, the oxybate or a pharmaceutically acceptable salt thereof containing about 6.135 g of GHB is administered per day. In some embodiments, the oxybate or a pharmaceutically acceptable salt thereof containing about 3.068 g of GHB is administered twice per day. In some embodiments, the oxybate or a pharmaceutically acceptable salt thereof containing about 2.045 g of GHB is administered three times per day.

In some embodiments, the oxybate or a pharmaceutically acceptable salt thereof containing about 7.362 g of GHB is administered per day. In some the embodiments, the oxybate or a pharmaceutically acceptable salt thereof containing about 3.681 g of GHB is administered twice per day. In some embodiments, the oxybate or a pharmaceutically acceptable salt thereof containing about 2.454 g of GHB is administered three times per day.

In some embodiments, the dose of the oxybate or a pharmaceutically acceptable salt thereof is described in terms of the amount of Equivalent Amount of GBA that is administered to the patient. The Equivalent Amount of GBA in a compositions may be calculated by the following formula:

$$Equivalent Amount of GBA = \frac{Amount of GHB in ( g ) \times 104.1 ( Formula Weight of GBA , \frac{g}{mol} )}{103.1 ( Formula Weight of GHB ( \frac{g}{mol} )}$$

In some embodiments, the oxybate or a pharmaceutically acceptable salt thereof containing about 0.826 g-7.434 g, about 1.652 g-8.26 g; about 2.478 g-7.847 g; or about 3.717 g-7.434 g of an Equivalent Amount of GBA is administered per day.

In some embodiments, the oxybate or a pharmaceutically acceptable salt thereof containing about 0.826 g of an Equivalent Amount of GBA is administered per day. In some embodiments, the oxybate or a pharmaceutically acceptable salt thereof containing about 0.413 g of an Equivalent Amount of GBA is administered twice per day. In some embodiments, the oxybate or a pharmaceutically acceptable salt thereof containing about 0.275 g of an Equivalent Amount of GBA is administered three times per day.

In some embodiments, the oxybate or a pharmaceutically acceptable salt thereof containing about 2.478 g of an Equivalent Amount of GBA is administered per day. In some embodiments, the oxybate or a pharmaceutically acceptable salt thereof containing about 1.239 g of an Equivalent Amount of GBA is administered twice per day. In some embodiments, the oxybate or a pharmaceutically acceptable salt thereof containing about 0.826 g of an Equivalent Amount of GBA is administered three times per day.

In some embodiments, the oxybate or a pharmaceutically acceptable salt thereof containing about 3.717 g of an Equivalent Amount of GBA is administered per day. In some embodiments, the oxybate or a pharmaceutically acceptable salt thereof containing about 1.859 g of an Equivalent Amount of GBA is administered twice per day. In some embodiments, the oxybate or a pharmaceutically acceptable salt thereof containing about 1.239 g of an Equivalent Amount of GBA is administered three times per day.

In some embodiments, the oxybate or a pharmaceutically acceptable salt thereof containing about 4.956 g of an Equivalent Amount of GBA is administered per day. In some embodiments, the oxybate or a pharmaceutically acceptable salt thereof containing about 2.478 g of an Equivalent Amount of GBA is administered twice per day. In some embodiments, the oxybate or a pharmaceutically acceptable salt thereof containing about 1.652 g of an Equivalent Amount of GBA is administered three times per day.

In some embodiments, the oxybate or a pharmaceutically acceptable salt thereof containing about 6.195 g of an Equivalent Amount of GBA is administered per day. In some embodiments, the oxybate or a pharmaceutically acceptable salt thereof containing about 3.098 g of an Equivalent Amount of GBA is administered twice per day. In some embodiments, the oxybate or a pharmaceutically acceptable salt thereof containing about 2.065 g of an Equivalent Amount of GBA is administered three times per day.

In some embodiments, the oxybate or a pharmaceutically acceptable salt thereof containing about 7.434 g of an Equivalent Amount of GBA is administered per day. In some embodiments, the oxybate or a pharmaceutically acceptable salt thereof containing about 3.717 g of an Equivalent Amount of GBA is administered twice per day. In some embodiments, the oxybate or a pharmaceutically acceptable salt thereof containing about 2.478 g of an Equivalent Amount of GBA is administered three times per day.

In some embodiments, the methods of the present disclosure comprise administering between 1 and 4.5 grams/day or between 6 and 10 grams/day of GHB. In some embodiments, the administered formulation comprises between 350-750 mg/ml or 450-550 mg/ml of GHB and has a pH between 6-10 or 6.5-8.

In some embodiments, the methods of the present disclosure comprise oral administration of the compositions or formulations comprising oxybate or a pharmaceutically acceptable salt thereof (disclosed herein) in a multiple dosage regimen. See U.S. Pat. No. 8,591,922, which is hereby incorporated by reference in its entirety for all purposes. In some embodiments, the multiple dosage regimen comprises one or more steps, as follows: (i) diluting an aqueous solution comprising about 500 mg/mL of the oxybate or a pharmaceutically acceptable salt thereof with an aqueous medium to provide a first dose of about 1-10 grams of the mixture of salts; (ii) orally administering the dose to a patient; (iii) diluting an aqueous solution comprising about 500 mg/mL of the oxybate or a pharmaceutically acceptable salt thereof to provide a second dose of about 1-10 grams of the oxybate or a pharmaceutically acceptable salt thereof; and (iv) orally administering to the patient the second dose. The dose administered to the patient can be between about 0.25-9.0 grams. (All volumes and numbers are presented as Na GHB equivalents).

In some embodiments, the composition comprising the oxybate or a pharmaceutically acceptable salt thereof is a liquid. In some embodiments, the concentration of the oxybate or a pharmaceutically acceptable salt thereof in the liquid is from 50 mg/mL-950 mg/mL, about 250 mg/mL-750 mg/mL, about 350 mg/mL-650 mg/mL, or about 450 mg/mL-550 mg/mL. In some embodiments, the concentration of the oxybate or a pharmaceutically acceptable salt thereof in the liquid is about 0.5 g/mL.

In one aspect, the present disclosure provides methods of treating the symptoms associated with fibromyalgia in a patient in need thereof with a slow wave sleep deficit (SWS) as determined by polysomnography (e.g. sleep electroencephalogram (EEG)) comprising administering a sustained release oxybate composition to the patient. In one aspect, the present disclosure provides methods of treating the symptoms associated with PTSD in a patient in need thereof with a slow wave sleep deficit (SWS) as determined by polysomnography (e.g. sleep electroencephalogram (EEG)) comprising administering a sustained release oxybate composition to the patient. In one aspect, the present disclosure provides methods of treating the symptoms associated with IBS or IBD in a patient in need thereof with a slow wave sleep deficit (SWS) as determined by polysomnography (e.g. sleep electroencephalogram (EEG)) comprising administering a sustained release oxybate composition to the patient. In some embodiments, the sustained release composition comprises a mixed salt oxybate. In some embodiments, the composition comprises a sustained release composition described in published U.S. Patent Publication Nos. 2020/0330393, 2018/0318222 and 2012/0076865, the contents of which is hereby incorporated by reference it entirety for all purposes.

As used herein, the term “controlled release” describes a formulation, such as, for example, a unit dosage form, that releases drug over a prolonged period of time. The controlled release compositions described herein may be provided as a unit dosage form suitable for oral administration. In each embodiment of the controlled release compositions described herein, the drug incorporated in such compositions may be selected from GHB and pharmaceutically acceptable salts, hydrates, tautomers, solvates and complexes of GHB. As is detailed herein, the controlled release dosage forms according to the present description include a controlled release component (also referred to as a controlled release “formulation”) and, optionally, an immediate release component (also referred to as an immediate release “formulation” or an immediate release “coating”). In specific embodiments, the controlled release dosage forms described herein may be formulated to deliver drug to the gastro-intestinal tract at desired rates of release or release profiles.

The present immediate release and controlled release formulations can be liquid or solid. These compositions can take the form of solutions, suspensions, troches, capsules, elixirs, suspensions, syrups, and the like. If solid, they can be tablets, buccal tablets or tabs, granules, pellets, beads, wafers, dry powders, capsules, etc. commonly known in the industry. See for example, “Remington's Pharmaceutical Sciences” 23^{rd} Edition.

The present disclosure provides, among other things, methods for administering oxybate or a pharmaceutically acceptable salt thereof (such as sodium oxybate or a mixed salt oxybate e.g., JZP-258) to a patient with fibromyalgia, PTSD, IBS or IBD wherein the identified patient exhibits no/low SWS (e.g. SWS of less than about 10%). Most patients administered according to the methods described herein do not have cardiovascular disease or another condition (for example, high risk of stroke, renal impairment or hypertension) that would indicate them for treatment with a low sodium oxybate composition. However, in some embodiments, the patient administered the oxybate or a pharmaceutically acceptable salt thereof (such as sodium oxybate or a mixed salt oxybate) is a patient at risk for the undesirable side effects related to high sodium intake. In some embodiments, the patient is in heart failure. In some embodiments, the patient is hypertensive. In some embodiments, the patient has renal impairment. In some embodiments, the patient is at risk for stroke. In some embodiments, the patient has no comorbid disease.

In some embodiments, a pharmacy management system may be required or preferred as part of a drug distribution program. For example, the present invention includes a method for distributing a drug containing GHB or a salt thereof to an approved pharmacy, the method comprising: (1) Identifying an approved pharmacy that has an established management system to dispense information concerning the risks associated with ingesting a MCT inhibitors concomitantly to said drug to patients that are prescribed said drug; (2) Providing said pharmacy with said information related to the risks; and (3) Authorizing distribution of said drug to said pharmacy, wherein said pharmacy dispenses the drug with said information when filling a prescription for said drug. The established management system may include an electronic alert to employees to dispense said information with said drug when prescriptions are filled. Such information may be dispensed in written form, for example in a brochure explaining the risks of concomitant ingestion of GHB and an MCT inhibitor such as diclofenac, valproate, or ibuprofen or combinations thereof. For example, the information dispensed with GHB may advise a patient of the potential for enhanced potency of GHB if the patient also takes valproate. Alternatively, or in addition thereto, the information dispensed with GHB may advise a patient of the potential for decreased potency of GHB if the patient also takes diclofenac. Such information may also be dispensed in verbal form. Distributors may maintain a directory of approved pharmacies, for example in a computer readable storage medium, to further ensure that GHB is dispensed only to patients who are advised of the additive effects.

A pharmacy management system of the present invention can be a REMS system as shown in U.S. Pat. Nos. 7,895,059; 7,797,171; 7,668,730 and 8,731,963. Warnings may be administered through the existing pharmacy management system as described in the patents above.

In some embodiments, the patient is treated is treated for excessive daytime sleepiness in patients identified herein. See U.S. Pat. Nos. 6,472,431; 6,780,889; 7,262,219; 8,263,650; 8,461,203, 8,591,922, 8,901,173, 9,132,107, 9,555,017, 9,795,567, 10,195,168, and U.S. Patent Publication Nos. 2020/0330393 and 2018/0263936 for example.

### Methods of Making

The oxybate or a pharmaceutically acceptable salt thereof, compositions and formulations may be prepared using methods that are known to those skilled in the art, including the methods described U.S. Pat. Nos. 8,591,922; 8,901,173; 9,132,107; 9,555,017; 10,195,168 and U.S. Publication No. 2018/0263936, which are hereby incorporated by reference.

### EXAMPLES

### Example 1

This study was a randomized, double-blind, placebo-controlled, parallel group study in subjects diagnosed with fibromyalgia.

Diagnosis and Main Criteria for Inclusion:

The study population for this study consisted of adult male and female subjects who were diagnosed with fibromyalgia according to the American College of Rheumatology (ACR) criteria for fibromyalgia (Wolfe et al 1990) and who had an average pain severity score above 4 (on a VAS scale of 0 to 10) at baseline. The ACR criteria included: presence of widespread pain for more than 3 months in all 4 quadrants of the body as well as along the spine and the elicitation of pain at 11 or more out of 18 anatomically defined tender points upon palpation with a force of 4 kg.

Subjects underwent screening assessments. Subjects who met the eligibility criteria underwent a drug withdrawal and washout period during which time pain medications and any treatment for fibromyalgia were gradually withdrawn. At the end of the 2-week baseline assessment period, subjects who continued to meet inclusion/exclusion criteria and who reported an average pain score above 4 (on a visual analog scale [VAS] of 0 to 10) during the last week of the baseline period were randomized to 1 of 3 treatment groups: Xyrem 4.5 g/night, Xyrem 6 g/night, or placebo (half of the subjects randomized to placebo received placebo matching 4.5 g and the other half received placebo matching 6 g).

Inclusion Criteria

Inclusion Criteria

Subjects were eligible for the trial if they:
1. Signed and dated an informed consent prior to beginning protocol required procedures.
2. Were willing and able to complete the entire trial as described in the protocol.
3. Were male or female >18 years of age
4. Met the American College of Rheumatology criteria for Fibromyalgia (Wolfe et al 1990):
   Widespread pain for at least 3 months, defined as the presence of all of the following:
   Pain on right and left sides of the body
   Pain above and below the waist
   Pain in the axial skeleton
   Pain on digital palpation with approximately 4 kg force in at least 11 of 18 tender point sites
5. (Study continuation) Had an average VAS pain score >4 on a scale of 0 to 10 as recorded in the patient diary the last week before Visit 4.
6. Were willing to discontinue opiates, benzodiazepines, anticonvulsants taken for pain, antidepressants, cyclobenzaprine (Flexeril), and/or tramadol (Ultram) until completion of the study.
7. Were willing to continue all preexisting nutritional and/or exercise regimens and/or behavioral, massage, acupuncture, physical or cognitive therapies on an unchanged, consistent and regular schedule throughout the course of the study.
8. Throughout the course of the trial agreed to use only acetaminophen or over-the-counter (OTC) non-steroidal anti-inflammatory drugs (NSAIDs) as rescue pain medications and to limit the dose to the following maxima:

| OTC | | Daily | |
|---|---|---|---|
| Analgesic | How taken | Maximum | |
| Acetaminophen | 650 mg or 1000 mg q4h | 4000 | mg |
| Ibuprofen | 200 mg q4h (if first dose is insufficient. | 1200 | mg |
| | ONE additional 200 mg dose may be | | |
| | taken one hour later, and adjust to 400 | | |
| | mg q4h) | | |
| Naproxen | 220 mg q 8 or 12 h OR 440 mg followed | 660 | mg |
| | by 220 mg 12 hours later | | |
| Ketoprofen | 12.5 mg q4h (if first dose is insufficient, | 75 | mg |
| | ONE additional 12.5 mg dose may be taken | | |
| | one hour later with dosing at 12.5 mg q4h | | |
| | thereafter) | | |

Each patient should take no more than one of these drugs during any given study day.
Formulations that add caffeine are not permitted.
Patients may, for cardiac protection, take no more than a single daily dose of 325 mg of aspirin. Any other use of aspirin is prohibited during this study.

9. Were willing to discontinue all prescription medications they were taking for fibromyalgia therapy.
10. Agreed to forego the ingestion of alcohol for the duration of the study.
11. Females may have been included who were surgically sterile, 2 years postmenopausal, or if of child-bearing potential, using a medically accepted method of birth control (eg, barrier method with spermicide, oral contraceptive, or abstinence) and agreed to continue use of this method for the duration of the trial.

Exclusion Criteria

Subjects who met any of the following criteria were excluded from the study: Had any of the following medical conditions:
1. Active rheumatic disease in addition to fibromyalgia, such as rheumatoid arthritis, osteoarthritis, or systemic lupus erythematosus which was causing pain of a severity and/or frequency sufficient to interfere with the evaluation of possible relief of fibromyalgia pain over the course of this trial.
2. Uncontrolled hypo- or hyper-thyroidism of any type.
3. Unstable cardiovascular, endocrine, neoplastic (excluding localized basal cell carcinoma), gastrointestinal, hematologic, hepatic, immunologic, metabolic, neurological, pulmonary, and/or renal disease which would place the patient at risk during the trial or compromise the objectives outlined in the protocol
4. A history of myocardial infarction within the last 6 months
5. On their screening PSG had an Apnea Index greater than 10 per hour or an AHI (Apnea Hypopnea Index) greater than 15 per hour. Note that patients with sleep apnea, treated with CPAP (continuous positive airway pressure), were not excluded if their indices were below these thresholds while sleeping with CPAP and they are compliant with CPAP therapy. One way to evaluate CPAP compliance was by review of built-in device usage logs.
6. Psychiatric disorders, major affective or psychotic disorders, or other problems that, in the investigator's opinion, would preclude the patient's participation and completion of this trial or compromise reliable representation of subjective symptoms.
7. If a patient had to discontinue antidepressant medication taken for depression, the investigator made an evaluation as to any risks from cessation of anti-depressant therapy. If, in the opinion of the investigator, a reasonable risk of resultant patient harm existed, the patient was excluded from study participation
8. A current or recent (within 1 year) history of a substance use disorder including alcohol abuse as defined by the DSM-IV (Diagnostic and Statistical Manual of Mental Disorders, Fourth Edition)
9. A clinically significant history of seizure disorder either past or present, a history of clinically significant head trauma (ie, concussion resulting in clinically significant loss of consciousness), migraine headaches or past invasive intracranial surgery, and were taking anticonvulsant medications
10. Succinic semialdehyde dehydrogenase deficiency
Had taken any of these therapies:
11. Gamma-hydroxybutyrate (sodium oxybate) in the 30 days prior to signing informed consent
12. Any investigational therapy in the 30 days prior to signing informed consent
13. Ever taken anticonvulsants to treat epilepsy or any other convulsions
Were unwilling to stop these therapies during the course of the trial:
14. Anticonvulsants prescribed solely for pain
15. All antidepressants (including but not limited to, tricyclic antidepressants (TCAs) or serotonin-selective reuptake inhibitors (SSRIs)). Subjects taking antidepressants who were willing to discontinue these medications may participate if they agreed to follow the investigators' recommended down-titration and washout program (5×the half-life of antidepressant).
16. Sleep aids such as hypnotics, tranquilizers, sedating antihistamines (non-sedating antihistamines are permitted during the trial), benzodiazepines.
Had any of the following exclusionary clinical laboratory results:
17. Serum creatinine greater than 2.0 mg/dL
18. Thyroid stimulating hormone (TSH) outside the normal range (for example, >4 μU/mL or <μU/mL are abnormal based on MedTox's [the central laboratory in use at this date] reference range)
19. Abnormal liver function tests (SGOT [AST] or SGPT [ALT] more than twice the upper limit of normal)
20. Elevated serum bilirubin (more than 1.5 times the upper limit of normal) Subjects known to have Gilbert's Disease (also known as Gilbert's Syndrome—causes hyperbilirubinemia with no known clinical consequence) were excepted from this exclusion criterion.
21. Pre-trial electrocardiogram (ECG) results demonstrating clinically significant arrhythmias, greater than a first degree AV block
22. Positive pregnancy test at any time during the trial Had any of the following exclusionary socio-economic factors:
23. Pending worker's compensation litigation or related other monetary settlements
24. Had an occupation that requires variable shift work or routine night shifts

Test Product, Dose, Mode of Administration, and Duration of Treatment:
Sodium oxybate 4.5 g, oral solution (9 mL) taken in 2 equally divided doses (4.5 mL added to 2 oz water) each night for 8 weeks.
Sodium oxybate 6 g, oral solution (12 mL) taken in 2 equally divided doses (6 mL added to 2 oz of water) each night for 8 weeks.

Reference Therapy, Dose and Mode of Administration:
Placebo (sodium citrate solution) 4.5 g, oral solution (9 mL) matching volume of Xyrem 4.5 g, taken in 2 equally divided doses (4.5 mL added to 2 oz water) each night.
Placebo (sodium citrate solution) 6 g, oral solution (12 mL) matching volume of Xyrem 6 g, taken in 2 equally divided doses (6 mL added to 2 oz water) each night.

Criteria for Evaluation:

Primary Efficacy:

Fibromyalgia Syndrome Composite Response:

The primary efficacy parameter was a binary composite parameter for the treatment of fibromyalgia syndrome. The proportion of subjects in each treatment group that met all 3 of the following response criteria was compared to assess the efficacy of Xyrem in response to fibromyalgia syndrome.
Pain Severity: as assessed by pain VAS data recorded 3 time a day by the subject in an electronic diary (eDiary). For pain VAS, a response was defined as a reduction in average pain of 20% or greater from baseline to Week 8.
Functionality (Fibromyalgia Impact Questionnaire [FIQ]): Change from baseline to a study visit was assessed. A response was defined as a reduction of 20% or greater in FIQ total score from baseline to Week 8.
Patient Global Impression of Change (PGIc): The subject's perception of the overall improvement in their fibromyalgia symptoms was assessed by means of the PGIc questionnaire completed at the end of study. A response to treatment was defined as a response of “Very much better” or “Much better” on the PGIc.

Secondary Efficacy:

Severity of Pain: the mean change from baseline to a study visit in pain severity (VAS pain). The proportion of responders for pain, defined as the proportion of subjects who had at least 20% reduction in pain severity from baseline to a study visit.

Fibromyalgia Impact Questionnaire: The mean change from baseline to a study visit in FIQ total score and subscale scores. The proportion of FIQ responders, defined as the proportion of subjects who had at least a 20% reduction in FIQ total score from baseline to a study visit.

Patient Global Impression of Change: This parameter was analyzed both as an ordinal categorical variable and a binary outcome variable. When PGIc was analyzed as an ordinal categorical variable, the mean score was analyzed. When PGIc was analyzed as a binary outcome variable, the proportion of subjects who had a response of “Very much better” or “Much better” was analyzed.

Subjective Assessments of Sleep and Fatigue:
Daytime Sleepiness: the mean change from baseline to a study visit in the Epworth Sleepiness Scale (ESS).
Sleep Patterns: the mean change from baseline to a study visit in the Jenkins Scale (JS).
Fatigue: the mean change from baseline to a study visit in the fatigue VAS score.
Functional Outcomes of Sleep Questionnaire (FOSQ): the mean changes from baseline to a study visit in FOSQ total score and subscale scores.

Objective Assessments of Sleep and Fatigue:
Clinical Polysomnography (PSG): Change from baseline to a study visit in various PSG variables.

Other Secondary Efficacy Assessments:
Clinical Global Impression of Change (CGIc): this parameter was analyzed both as an ordinal categorical variable and a binary outcome variable. When CGIc was analyzed as an ordinal categorical variable, the mean score was analyzed. When CGIc was analyzed as a binary outcome variable, the proportion of subjects who had a response of “Very much improved” or “Much improved” was analyzed.
Tender Point Count and Index: the mean change in Tender Point Count (TC) and Tender Point Index (TI) from baseline to a study visit.
Short Form-36 Questionnaire (SF-36): The mean change from baseline to a study visit in Physical (PCS), Mental (MCS) Component Summary measures, and each subscale score.
Pain (rescue medication use): Change from baseline to a study visit in the number of days during the 1-week period prior to the visit in which rescue medication was required and the proportion of subjects who did not use rescue medication.

Safety:

The safety endpoints included the incidence of adverse events (AEs), laboratory test results (hematology & biochemistry at baseline and Week 8), vital signs and body weight results (hematology & biochemistry at baseline and Week 8), vital signs and body weight (baseline and Week 8), investigator's assessment of electrocardiogram (ECG) results (screening and Week 8), study medication exposure, use of concomitant medications, physical examination results (screening and Week 8).

Statistical Methods

General Considerations:

The primary analysis population for efficacy parameters was the intent-to-treat (ITT) population, which included all randomized subjects. The per-protocol (PP) population was a subset of the ITT population and included all subjects who completed the study per the protocol; the PP population was used for a secondary analysis of the primary efficacy endpoint. The all-treated population consisted of all subjects who received at least one dose of study drug and was the analysis population for the safety analyses.

| | | Sodium Oxybate | Sodium Oxybate | |
|---|---|---|---|---|
| | Placebo | (4.5 g/night) | (6 g/night) | Total |
| ITT population | 66 | 62 | 67 | 195 |
| PP population | 46 | 44 | 38 | 128 |
| Safety (all | 65 | 60 | 67 | 192 |
| treated) | | | | |

Fibromyalgia Syndrome Composite Response: The primary efficacy parameter was analyzed using the chi-square test at Week 8 using LOCF data from the ITT population. If an overall significant treatment difference was detected (0.05 significance level), then pairwise comparisons between each Xyrem group and placebo were conducted at the same significance level to identify effective doses. Analyses were also performed using baseline observation carried forward (BOCF) and observed data at Week 8 for the ITT and PP populations.

Secondary Efficacy Endpoints: Analysis of variance (ANOVA) models were used to analyze the change from baseline for the secondary efficacy parameters. A responder analysis was performed for FIQ, overall pain VAS, PGIc, CGIc, and the proportion of subjects who did not use rescue medication at each study visit using a chi-square test. The Cochran-Mantel-Haenszel test was used to analyze ordinal categorical variables (Clinical Global Impression of severity [CGIs] at baseline, PGIc, and CGIc). If a significant overall treatment difference was found (0.05 significance level), then pairwise comparisons between each Xyrem group and placebo were conducted at the same 0.05 level. The significance of the mean change from baseline within each dose group was determined using a paired t-test. For change from baseline, if parametric assumptions were violated, then ANOVA on ranks was performed for overall treatment difference and pairwise comparisons with placebo. Within-treatment change was analyzed with the Wilcoxon signed rank test. All of the secondary efficacy parameters (except the PSG variables) were analyzed at the time points where they were assessed using LOCF, BOCF, and observed data in the ITT population. PSG parameters were analyzed using observed data only in the ITT population.

### Example 2

This study was carried out to evaluate whether the % of slow wave sleep (specifically, a SWS % deficit) predicts a differentially greater response to oxybate.

An analysis of the polysomnographic (PSG) data from patients in the trial described in Example 1 was conducted to evaluate the relationship between slow wave (stage 3/4) sleep levels and oxybate response in patients with fibromyalgia.

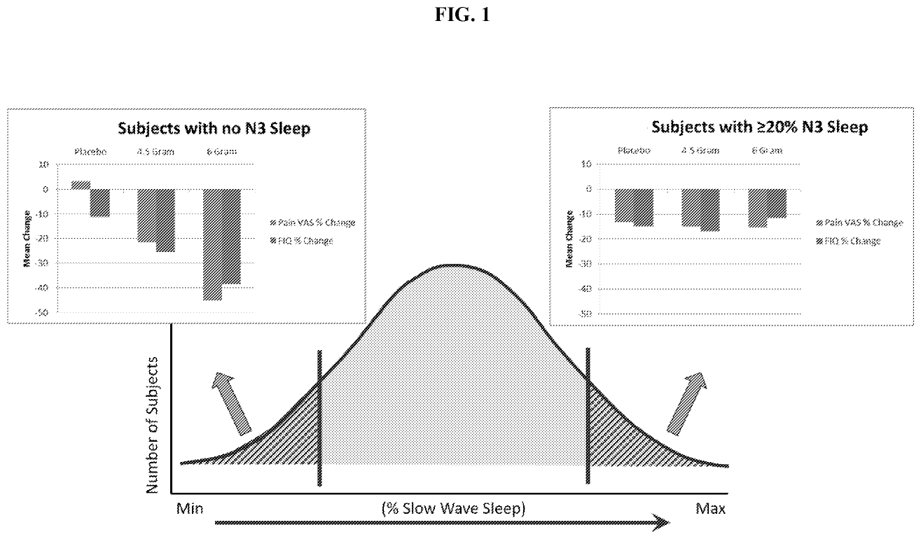

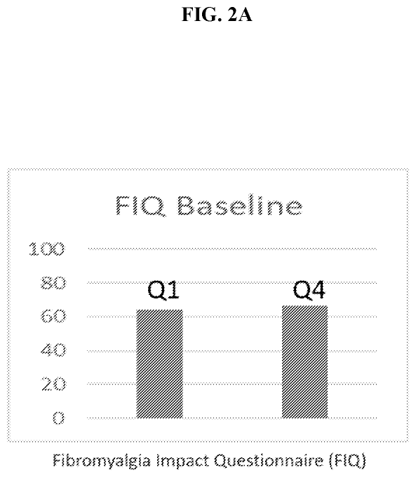

As shown in FIG. 1, patients that had less than about 1% slow wave sleep either at baseline or screening exhibited a dose-dependent response to oxybate and significant improvements in pain visual analog scale (PVAS) and Fibromyalgia Impact Questionnaire (FIQ) scores compared with placebo-treated control patients. In contrast, patients exhibiting ≥20% slow wave sleep prior to treatment did not exhibit a dose-dependent response to oxybate and changes in PVAS and FIQ scores did not differ significantly between patients receiving 4 g or 6 g sodium oxybate and the placebo-treated control patients. These results provided an initial indication that patients with no/low SWS experienced differentially greater treatment effect than fibromyalgia patients who are not classified as having no/low SWS.

Patients from the clinical trial described in Example 1 were then split into quartiles based on time spent in stage 3/4 sleep at baseline (Table 1), and the relationships between slow wave sleep and disease severity and oxybate response was analyzed in patients from Quartiles 1 and 4 to compare participants with extremes of SWS amounts.

TABLE 1
Patients Classified By Time Spent in Stage 3/4 Sleep at Baseline.

| Quartile | Cut-off (min) | Placebo N | 4.5 g N | 6 g N | Female/Male |
|---|---|---|---|---|---|
| 1 | <13.5 | 15 | 17 | 13 | 42/3 |
| 2 | 13.5-<38.75 | 16 | 15 | 15 | 41/5 |
| 3 | 38.75-<63 | 19 | 14 | 12 | 43/2 |
| 4 | 63+ | 11 | 14 | 21 | 46/0 |

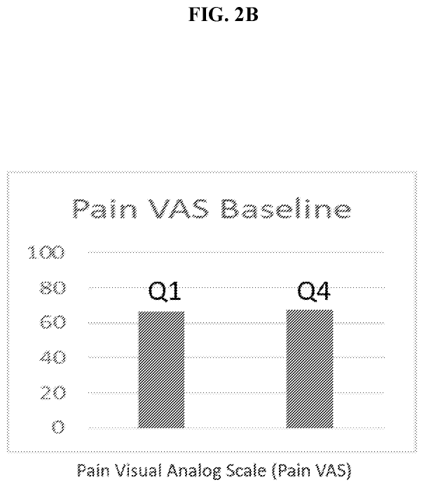

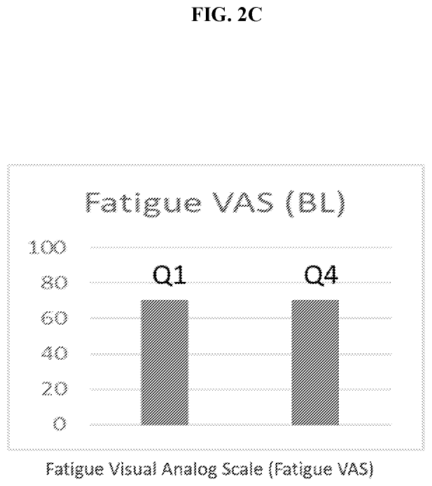

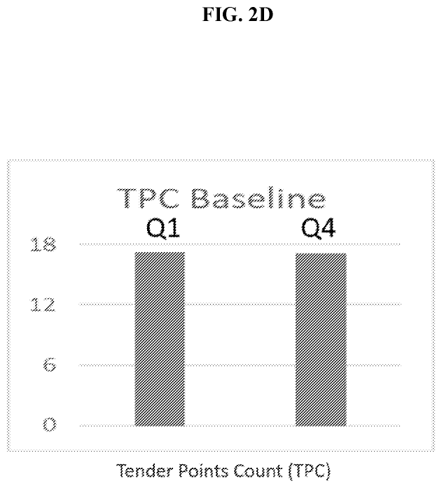

As shown in FIG. 2A-FIG. 2D there is no association between the level of slow wave sleep and disease severity.

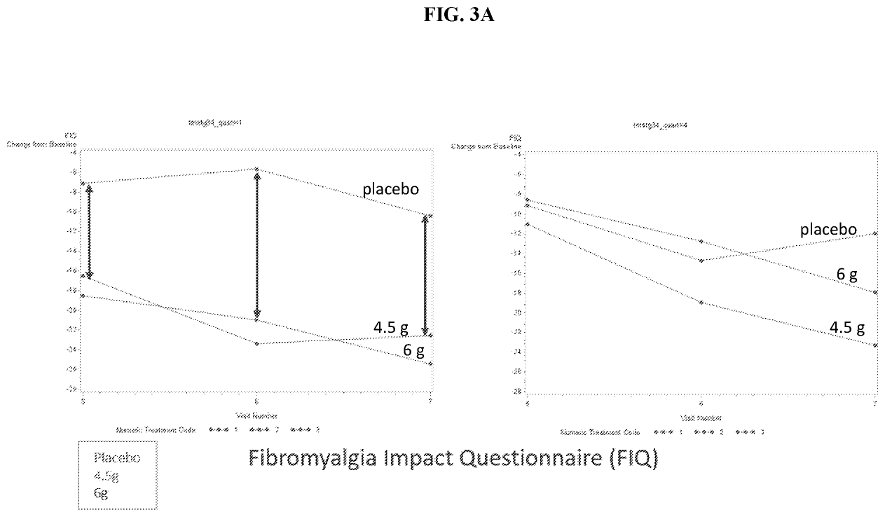

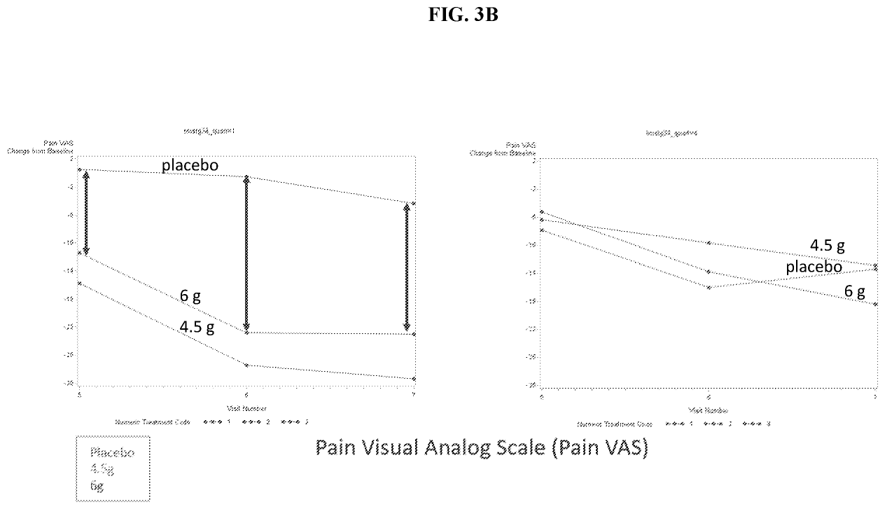

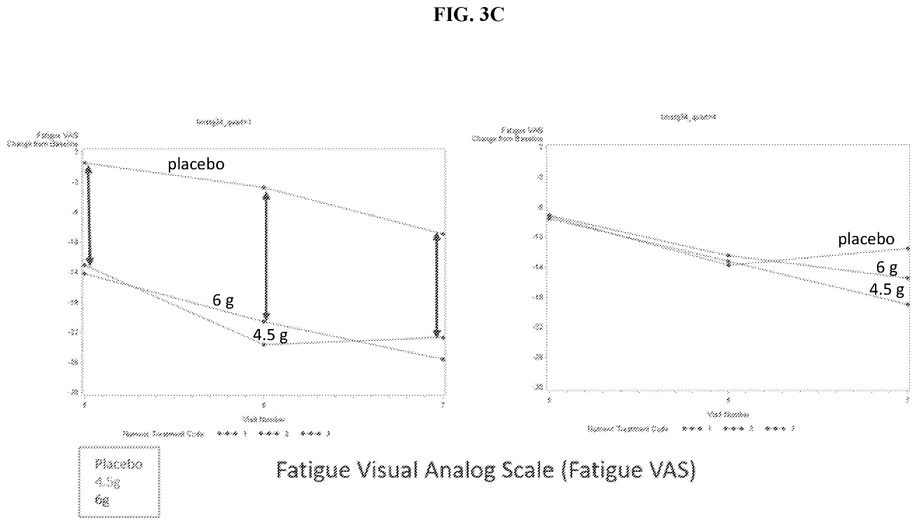

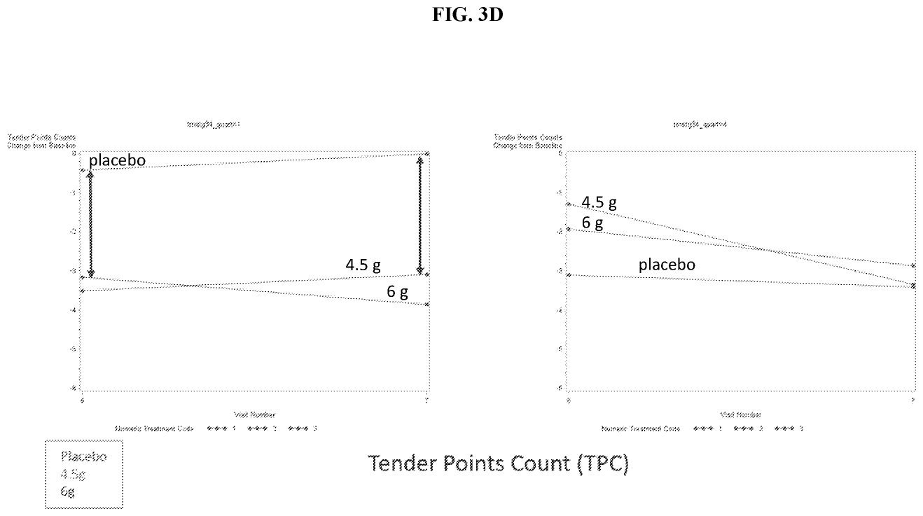

As shown in FIG. 3A-FIG. 3D low baseline slow wave sleep (quartile 1) was associated with improved oxybate response. These results indicate that patients with no/low SWS experienced differentially greater treatment effect than fibromyalgia patients who are not classified as having no/low SWS.

### Example 3

A study was carried out to evaluate Slow Wave Sleep as an enrichment indicator for Fibromyalgia and PTSD in patients from the Morpheus Clinical Practice PSG Dataset who were diagnosed with fibromyalgia or PTSD or had clinical presentation suggestive of PTSD and fibromyalgia. The Morpheus Clinical Sleep Database contains approximately 600 k patient PSGs.

The database was queried for diagnoses of fibromyalgia or PTSD or signs of fibromyalgia or PTSD. Signs for fibromyalgia included self-reported chronic pain with disrupted sleep in the absence of obstructive sleep apnea (OSA), and Periodic Limb Movement Disorder (PLMD). Signs for PTSD included a) veteran patients that report disrupted sleep due to disturbing dreams in the absence of OSA, PLMD, etc.; and b) patients from general clinics with a diagnosis of anxiety, disrupted sleep, disturbing dreams, etc. in the absence of OSA, or PLMD. The “fibromyalgia” and “PTSD” groups were subsequently reviewed by human researchers to corroborate/rule out the diagnosis in the physicians records (EMR). The target was to query database for 250 subjects with each disorder and chart review for ˜50 “best cases” for each disorder. The fibromyalgia patient population comprised females except for 2 males, whereas the PTSD patient population was more evenly distributed across sexes. Average ages are similar, with few patients under 40. There is high heterogeneity in the amount of SWS across both patient populations. The variability in SWS for the fibromyalgia patient population was 0-29%, despite mean % SWS being consistent with the reference data (16.3% vs˜17%). The variability in SWS for the PTSD patient population was despite same mean % SWS being consistent with the reference data (19% vs 17%).

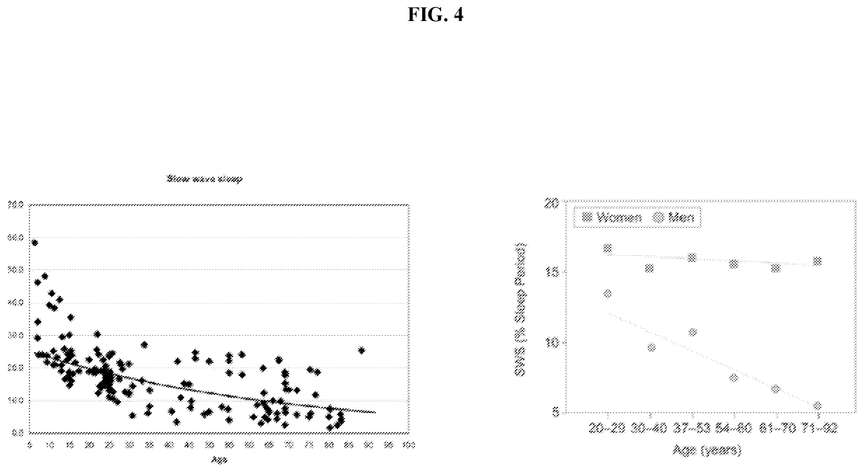

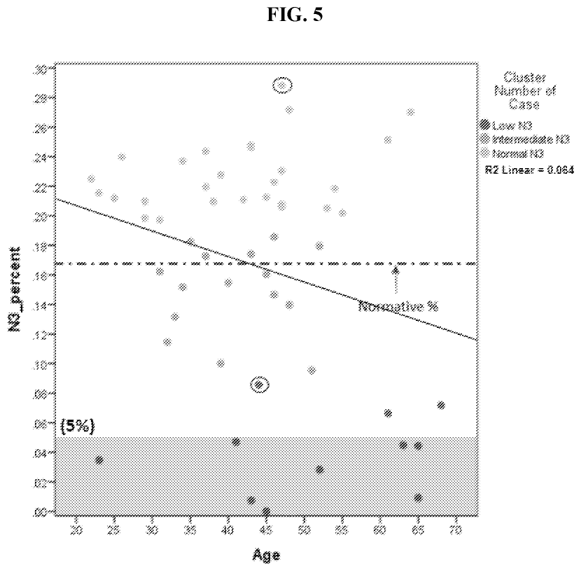

As shown in FIG. 5, eight subjects (15%) in the “fibromyalgia” population showed ≤5% slow wave sleep which correlated with the SWS distribution observed in Example 2.

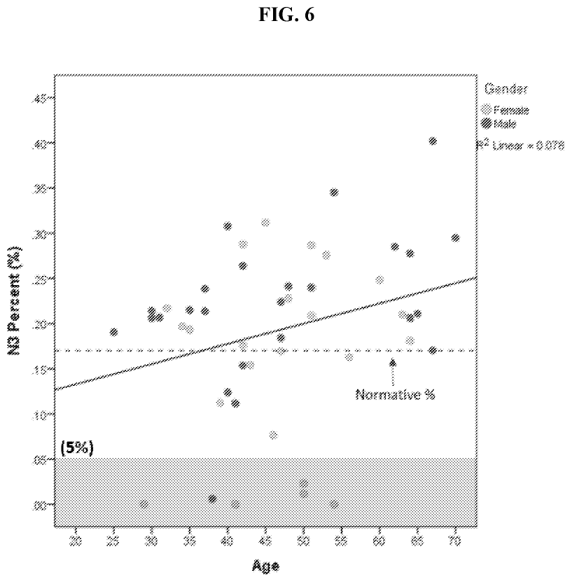

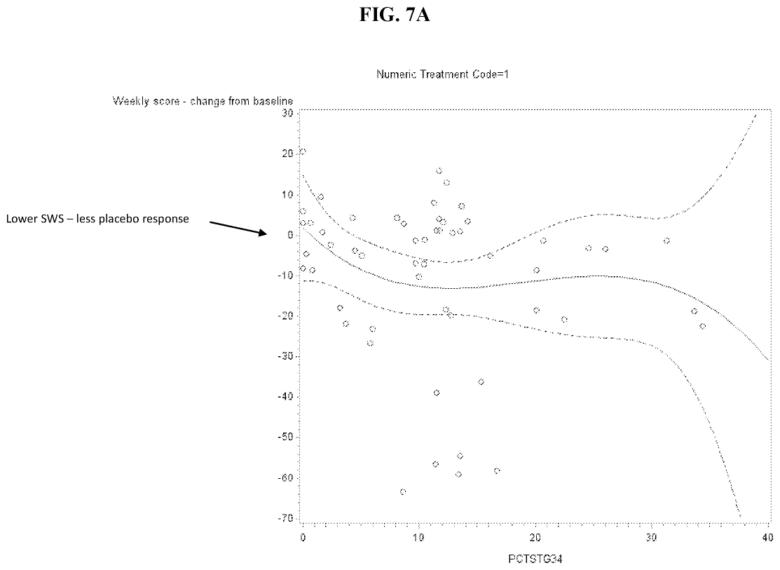

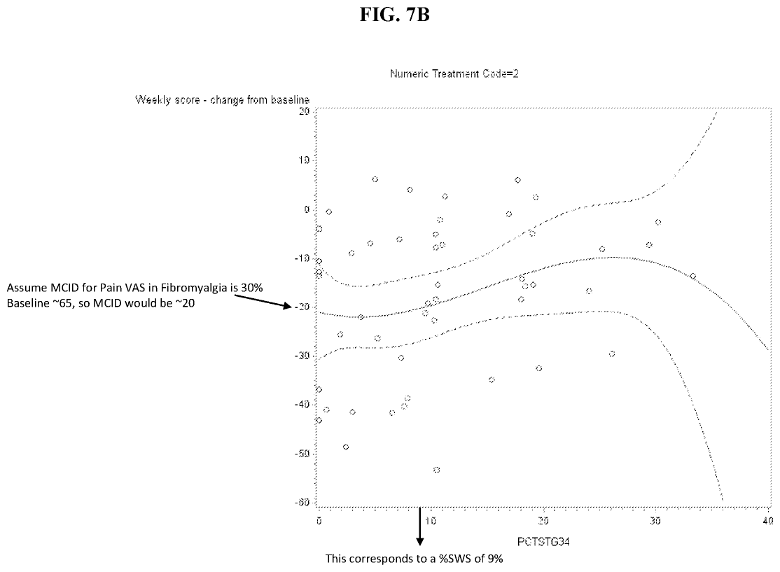

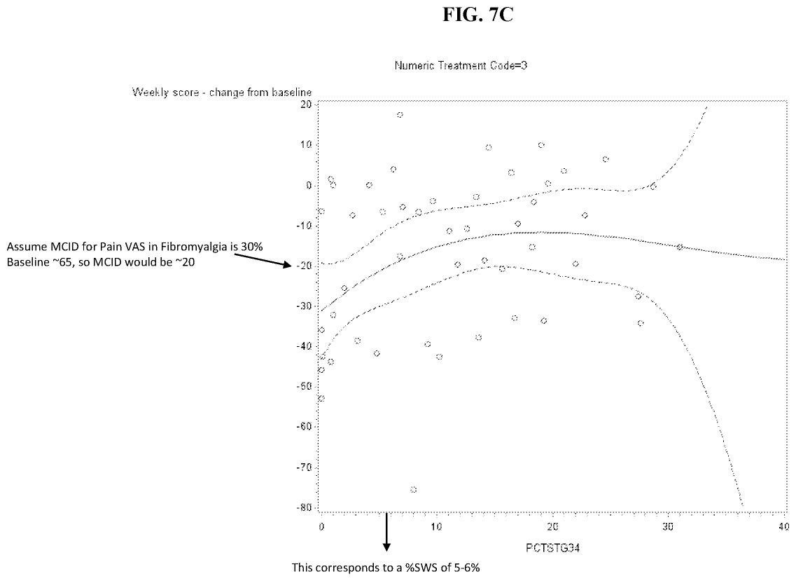

As shown in FIG. 6, six subjects (12.5%) of the “PTSD” population show ≤5% slow wave sleep. Five of the subjects with 5% slow wave sleep were female. The high level of clinical overlap between fibromyalgia, PTSD, IBS and IBD suggests overlapping pathophysiology indicating that both fibromyalgia and PTSD patients with a SWS deficit benefit from oxybate therapy.

These results indicate that (1) 12-15% of fibromyalgia and PTSD patients have <5% SWS, and (2) that these patients preferentially respond to oxybate treatment.

Based on these analyses, SWS is likely a useful enrichment indicator for oxybate response in PTSD and fibromyalgia.

1. A method of treating the symptoms associated with fibromyalgia in a patient in need thereof, the method comprising:
(a) identifying a patient with fibromyalgia with a slow wave sleep (SWS) deficit as determined by sleep polysomnography; and
(b) administering a therapeutically effective amount of oxybate or a pharmaceutically acceptable salt thereof to the patient.

2. The method of claim 1, wherein the identified patient exhibits SWS of less than about 10%.

3. The method of claim 1, wherein the identified patient exhibits SWS of less than about 5%.

4. The method of any one of claims 1-3, wherein the administration provides an improvement in the patient's Fibromyalgia Impact Questionnaire (FIQ) score or Tender Points Index (TPI) score compared to prior to the treatment.

5. The method of any one of claims 1-4, wherein the administration provides an improvement in the patient's Pain Visual Analogue Scale (P-VAS) score compared to prior to the treatment.

6. The method of any one of claims 1-5, wherein the administration provides an improvement in the patient's Fatigue Visual Analogue Scale (F-VAS) score compared to prior to the treatment.

7. The method of any one of claims 1-6, wherein the administration provides an improvement in the patient's Tender Points Count (TPC) score compared to prior to the treatment.

8. The method of any one of claims 1-7, wherein the oxybate or a pharmaceutically acceptable salt thereof is a mixed salt oxybate.

9. The method of claim 8, wherein the mixed salt oxybate comprises sodium oxybate, potassium oxybate, magnesium oxybate and calcium oxybate, and wherein the mixed salt oxybate comprises about 5%-40% sodium oxybate (wt/wt %).

10. The method of claim 8, wherein the mixed salt oxybate comprises about 5%-40% of sodium oxybate (wt/wt %), about 10%-40% of potassium oxybate (wt/wt %), about 5%-30% of magnesium oxybate (wt/wt %), and about 20%-80% of calcium oxybate (wt/wt %).

11. The method of claim 8, wherein the mixed salt oxybate comprises about 8% mol. equiv. of sodium oxybate, about 23% mol. equiv. of potassium oxybate, about 21% mol. equiv. of magnesium oxybate and about 48% mol. equiv. calcium oxybate.

12. The method of any one of claims 1-7, wherein the oxybate or a pharmaceutically acceptable salt comprises sodium oxybate.

13. The method of any one of claims 1-12, wherein about 4.5 g-6.0 g of the oxybate or a pharmaceutically acceptable salt thereof is administered per day.

14. The method of claim 13, wherein about 4.5 g of the oxybate or a pharmaceutically acceptable salt thereof is administered per day.

15. The method of claim 13, wherein about 6.0 g of the oxybate or a pharmaceutically acceptable salt thereof is administered per day.

16. A method of treating the symptoms associated with post-traumatic stress disorder (PTSD) in a patient in need thereof, the method comprising:
(a) identifying a patent with PTSD with a SWS deficit as determined by sleep EEG; and
(b) administering a therapeutically effective amount of oxybate or a pharmaceutically acceptable salt thereof.

17. The method of claim 16, wherein the identified patient exhibits SWS of less than about 10%.

18. The method of claim 16 wherein the identified patient exhibits SWS of less than about 5%.

19. The method of claim 18, wherein the administration provides an improvement in the patient's sleep symptoms associated with PTSD compared to prior to the treatment.

20. The method of claim 18, wherein the administration provides an improvement in the patient's insomnia, nightmares, somniphobia, or other sleep-related symptoms of PTSD compared to prior to the treatment.

21. The method of any one of claims 16-20, wherein the oxybate or a pharmaceutically acceptable salt thereof is a mixed salt oxybate.

22. The method of claim 21, wherein the mixed salt oxybate comprises sodium oxybate, potassium oxybate, magnesium oxybate and calcium oxybate, and wherein the mixed salt oxybate comprises about 5%-40% sodium oxybate (wt/wt %).

23. The method of claim 21, wherein the mixed salt oxybate comprises about 5%-40% of sodium oxybate (wt/wt %), about 10%-40% of potassium oxybate (wt/wt %), about 5%-30% of magnesium oxybate (wt/wt %), and about 20%-80% of calcium oxybate (wt/wt %).

24. The method of claim 21, wherein the mixed salt oxybate comprises about 8% mol. equiv. of sodium oxybate, about 23% mol. equiv. of potassium oxybate, about 21% mol. equiv. of magnesium oxybate and about 48% mol. equiv. calcium oxybate.

25. The method of any one of claims 16-20, wherein the oxybate or a pharmaceutically acceptable salt thereof comprises sodium oxybate.

26. A method of treating the symptoms associated with irritable bowel disease (IBD) or irritable bowel syndrome (IBS) in a patient in need thereof, the method comprising:
(a) identifying a patent with IBD or IBS with a SWS deficit as determined by sleep EEG; and
(b) administering a therapeutically effective amount of oxybate or a pharmaceutically acceptable salt thereof.

27. The method of claim 26, wherein the identified patient exhibits SWS of less than about 10%.

28. The method of claim 26, wherein the identified patient exhibits SWS of less than about 5%.

29. The method of claim 28, wherein the administration provides an improvement in the patient's sleep symptoms associated with IBS or IBD compared to prior to the treatment.

30. The method of claim 28, wherein the administration provides an improvement in the patient's insomnia associated with IBS or IBD compared to prior to the treatment.

31. The method of any one of claims 26-30, wherein the oxybate or a pharmaceutically acceptable salt thereof is a mixed salt oxybate.

32. The method of claim 31, wherein the mixed salt oxybate comprises sodium oxybate, potassium oxybate, magnesium oxybate and calcium oxybate, and wherein the mixed salt oxybate comprises about 5%-40% sodium oxybate (wt/wt %).

33. The method of claim 31, wherein the mixed salt oxybate comprises about 5%-40% of sodium oxybate (wt/wt %), about 10%-40% of potassium oxybate (wt/wt %), about 5%-30% of magnesium oxybate (wt/wt %), and about 20%-80% of calcium oxybate (wt/wt %).

34. The method of claim 31, wherein the mixed salt oxybate comprises about 8% mol. equiv. of sodium oxybate, about 23% mol. equiv. of potassium oxybate, about 21% mol. equiv. of magnesium oxybate and about 48% mol. equiv. calcium oxybate.

35. The method of any one of claims 26-30, wherein the oxybate or a pharmaceutically acceptable salt thereof comprises sodium oxybate.

36. The method of claim 1-35, wherein the method comprises:
(a) administering an initial daily dose of the oxybate or a pharmaceutically acceptable salt thereof to the patient and
(b) titrating the dose to provide a therapeutically effective amount of the oxybate or a pharmaceutically acceptable salt thereof.

37. The method of claim 36, wherein the initial daily dose is from about 0.5 g to about 4.5 g of the oxybate or a pharmaceutically acceptable salt thereof is administered per day.

38. The method of claim 37, wherein the initial daily dose is about 4.5 g of the oxybate or a pharmaceutically acceptable salt thereof is administered per day.

39. The method of any one of claims 36-38, wherein the titration step (b) comprises administering ascending doses of the oxybate or a pharmaceutically acceptable salt thereof.

40. The method of claim 39, wherein dose is increased by about 0.5 g to 1.5 g per week.

41. The method of any one of claims 36-38, wherein the titration step (b) comprises administering descending doses of the oxybate or a pharmaceutically acceptable salt thereof.

42. The method of claim 41, wherein dose is decrease by about 0.5 g to 9.0 g per week.

43. The method of any one of claims 36-42, wherein the titration step (b) comprises switching a patient from a once a day dose to a twice a day dose of the oxybate or a pharmaceutically acceptable salt thereof.

44. The method of any one of claims 36-42, wherein the titration step (b) comprises switching a patient from a twice a day dose to a three times a day dose of the oxybate or a pharmaceutically acceptable salt thereof.

45. The method of any one of claims 36-42, wherein the titration step (b) comprises switching a patient from a twice a day dose to a once a day dose of the oxybate or a pharmaceutically acceptable salt thereof.

46. The method of any one of claims 36-42, wherein the titration step (b) comprises switching a patient from a three times a day dose to a twice a day dose of the oxybate or a pharmaceutically acceptable salt thereof.

47. The method of any one of claims 36-46, wherein the titration step (b) is from about 1 week to about 14 weeks.

48. The method of any one of claims 1-12 and 16-47, wherein about 0.25 g-10.0 g, 2.0 g-10.0 g; about 3.0 g-9.5 g; or about 4.5 g-9.0 g of the oxybate or a pharmaceutically acceptable salt thereof is administered per day.

49. The method of claim 48, wherein the oxybate or a pharmaceutically acceptable salt thereof is administered twice per day.

50. The method of claim 48, wherein the oxybate or a pharmaceutically acceptable salt thereof is administered once per day.

51. The method of any one of claims 1-12 and 16-50, wherein about 4.5 g of the oxybate or a pharmaceutically acceptable salt thereof is administered per day.

52. The method of claim 51, wherein about 2.25 g of the oxybate or a pharmaceutically acceptable salt thereof is administered twice per day.

53. The method of any one of claims 1-12 and 16-50, wherein about 6.0 g of the oxybate or a pharmaceutically acceptable salt thereof is administered per day.

54. The method of claim 53, wherein about 3.0 g of the oxybate or a pharmaceutically acceptable salt thereof is administered twice per day.

55. The method of any one of claims 1-12 and 16-50, wherein about 7.5 g of the oxybate or a pharmaceutically acceptable salt thereof is administered per day.

56. The method of claim 55, wherein about 3.75 g of the oxybate or a pharmaceutically acceptable salt thereof is administered twice per day.

57. The method of any one of claims 1-12 and 16-50, wherein about 9.0 g of the oxybate or a pharmaceutically acceptable salt thereof is administered per day.

58. The method of claim 57, wherein about 4.5 g of the oxybate or a pharmaceutically acceptable salt thereof is administered twice per day.

59. The method of any one of claims 1-58, wherein the oxybate or a pharmaceutically acceptable salt thereof composition is a liquid.

60. The method of claim 59, wherein the concentration of the oxybate or a pharmaceutically acceptable salt thereof in the liquid is from 350 mg/ml-650 mg/ml, or about 450 mg/ml-550 mg/ml.

61. The method of claim 49, wherein the concentration of the oxybate or a pharmaceutically acceptable salt thereof in the liquid is about 0.5 g/mL.

62. The method of any one of claims 1-61, wherein the oxybate or a pharmaceutically acceptable salt thereof is administered at bedtime.

63. The method of any one of claims 1-62, wherein the oxybate or a pharmaceutically acceptable salt thereof is administered at bedtime and about 2.5 h-4 h after the bedtime administration.

64. The method of any one of claims 1-63, wherein the administration provides increased percentage of SWS compared to prior to the administration.

65. The method of any one of claims 1-64, wherein the administration provides increased percentage of REM sleep compared to prior to the administration.

66. The method of any one of claims 1-65, wherein the administration provides improved sleep quality as determined by the Pittsburgh Sleep Quality Index (PSQI).
